(19) United States
(12) Patent Application Publication
(10) Pub. No.: US 2021/0319912 A1
(43) Pub. Date: Oct. 14, 2021

(54) SYSTEMS AND METHODS FOR CONTACT AVOIDANCE FOR PREVENTING EPIDEMICS

(71) Applicant: Matrics2, Inc., Warba, MN (US)

(72) Inventors: William R. Bandy, Gambrills, MD (US); Michael R. Arneson, Warba, MN (US)

(21) Appl. No.: 17/228,116

(22) Filed: Apr. 12, 2021

Related U.S. Application Data
(60) Provisional application No. 63/101,011, filed on Apr. 13, 2020.

(51) Int. Cl. G16H 50/80; G16H 10/60; G16H 50/30; G16H 10/40; G16H 40/20; G06F 16/23; G10L 25/51; G06F 3/16; H04L 29/08; G08B 7/06; G01S 15/74

(52) U.S. Cl. CPC G16H 50/80; G16H 10/60; G16H 50/30; G16H 10/40; G16H 40/20; G01S 15/74; G10L 25/51; G06F 3/165; H04L 67/141; G08B 7/06; G06F 16/2379

(57) ABSTRACT

Embodiments described herein are directed to a contact avoidance system for preventing epidemics. A database maintains a record for each person enrolled in the system. The record comprises a risk level identifier that indicates a disease infection risk for the user. When devices carried by users are proximate to each other, the devices exchange identifiers that uniquely and anonymously identify the users. At least one device provides the identifier received thereby to the database, which retrieves the record associated with the identifier and determines a risk level for the user associated with the identifier. The risk level is returned to the device, which issues an alert if the risk level indicates that the other user is or may be infected with a disease. The database also updates the risk level associated with users that were determined to be proximate to a user determined to be infected with the disease.

### CROSS-REFERENCE TO RELATED APPLICATION(S)

This application claims priority to U.S. Provisional Patent Application No. 63/101,011 entitled “STOPPING EPIDEMICS OF NOVEL PATHOGENS WITH INFECTED PERSON ALERT SYSTEM,” and filed on Apr. 13, 2020, the entirety of which is incorporated by reference herein.

### BACKGROUND

The novel coronavirus (i.e., COVID-19) that ravaged the world in 2020 caught the world unprepared for an epidemic that cost countless lives and trillions of dollars. It exposed many deficiencies in how to stop an epidemic, a primary one being the lack of data to enable contagion prevention.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Methods, systems, apparatuses, and computer-readable storage mediums described herein for a contact avoidance system for preventing epidemics. For example, the system may comprise computing devices associated with users and a central database. The central database maintains a record for each person enrolled in the IPAS. The record comprises an infection risk level identifier that indicates a risk that the user has been infected with a particular disease. The record is also associated with records associated with other users that were determined to be in close proximity to the user (e.g., within 30 feet). Each computing device enrolled in the IPAS comprises an IPAS application. The IPAS application executing on a first computing device is configured to detect whether a second computing device enrolled in the IPAS is in close proximity therewith. Upon detecting as such, the computing devices exchange identifiers that anonymously and uniquely identify each other. The IPAS application executing on at least one of the computing devices provides the identifier received thereby to the central database. The central database analyzes the record associated with the received identifier (i.e., the detected user's record) and determines the risk level of the user associated with the identifier. The database provides the risk level to the IPAS application, and the IPAS application then provides an alert (e.g., an audible, visual or vibrating alert) on the user's device based on the determined risk level, letting the user know that he is in close proximity with a user that may be infected with the particular disease. The user may then maintain a safe distance from that other person, thereby avoiding contact with that person and preventing the disease from spreading.

The central database may also update the risk level associated with a particular record based on the risk levels of the other records associated therewith. For example, if a particular record has a risk level that indicates that the user has a particular disease, records associated therewith may also be updated with a new risk level. The new risk level may be dependent on the strength of interactions between the users associated with such records (e.g., the time the users spent with each other, the distance between the users between interactions, etc.). The IPAS application associated with a particular user may periodically communicate with the central database to determine the user's risk level and alert the user accordingly.

Further features and advantages, as well as the structure and operation of various example embodiments, are described in detail below with reference to the accompanying drawings. It is noted that the example implementations are not limited to the specific embodiments described herein. Such example embodiments are presented herein for illustrative purposes only. Additional implementations will be apparent to persons skilled in the relevant art(s) based on the teachings contained herein.

### BRIEF DESCRIPTION OF THE DRAWINGS/FIGURES

The accompanying drawings, which are incorporated herein and form a part of the specification, illustrate example embodiments of the present application and, together with the description, further serve to explain the principles of the example embodiments and to enable a person skilled in the pertinent art to make and use the example embodiments.

FIG. 1A depicts a graph showing the percentage of the United States population infected by an uncontrolled epidemic of a novel pathogen and without self-quarantine.

FIG. 1B depicts a graph showing the number of deaths from the epidemic with and without self-quarantine.

FIG. 1C depicts a graph showing the number of people infected per day with and without self-quarantine.

FIG. 1D depicts a graph showing the percentage of the United States population infected by an epidemic with growth controlled by a shutdown and with and without an infected person alert system in accordance with embodiments described herein.

FIG. 1E depicts a graph showing the number of deaths from the epidemic controlled by a shutdown with and without an infected person alert system in accordance with embodiments described herein.

FIG. 1F depicts a graph showing the number of people infected per day from the epidemic controlled by a shutdown with and without an infected person alert system in accordance with embodiments described herein.

FIG. 1G depicts a graph showing the ramp up of an infected person alert system and the successful re-opening of the economy compared to a failed attempt to re-open without the infected person alert system in accordance with embodiments described herein.

FIG. 1H depicts a graph showing the benefit of deploying an infected person alert system in accordance with embodiments described herein.

FIG. 11 depicts a graph showing the number of deaths when deploying an infected person alert system in accordance with embodiments described herein.

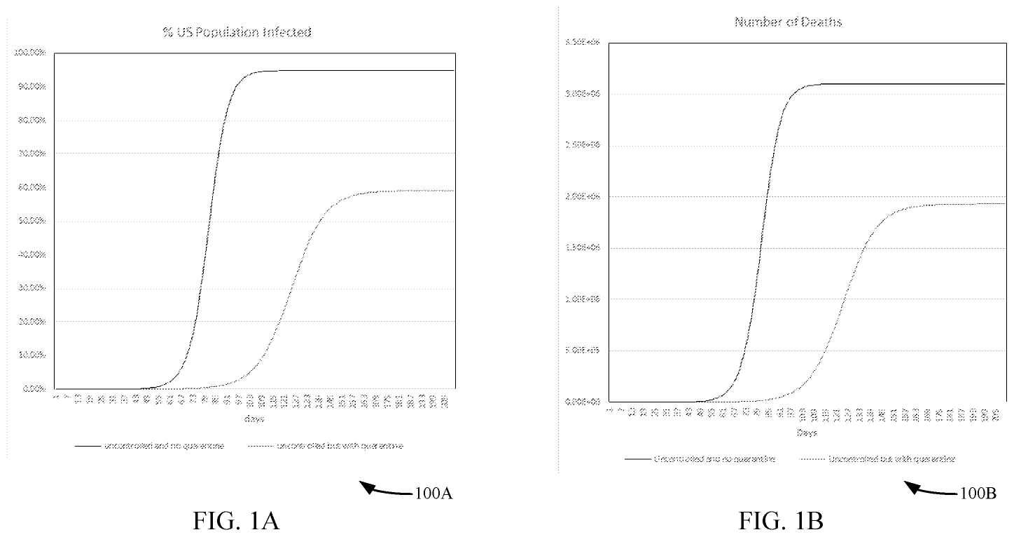

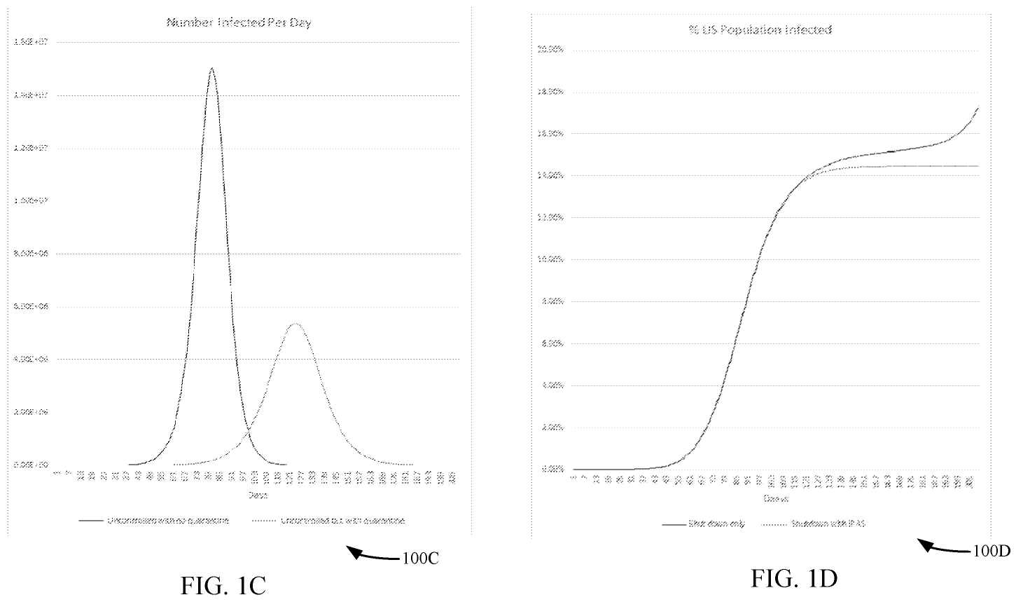

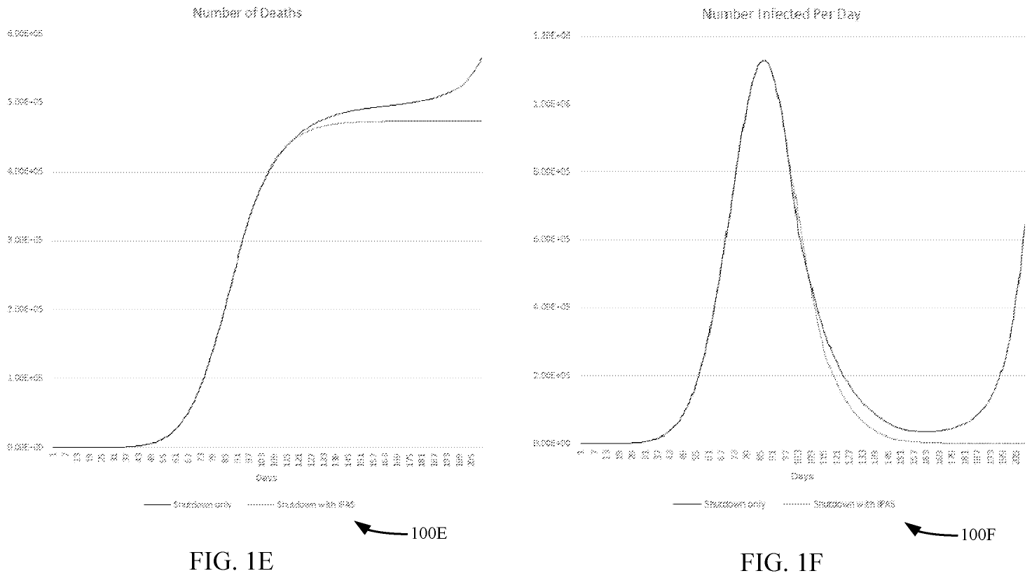

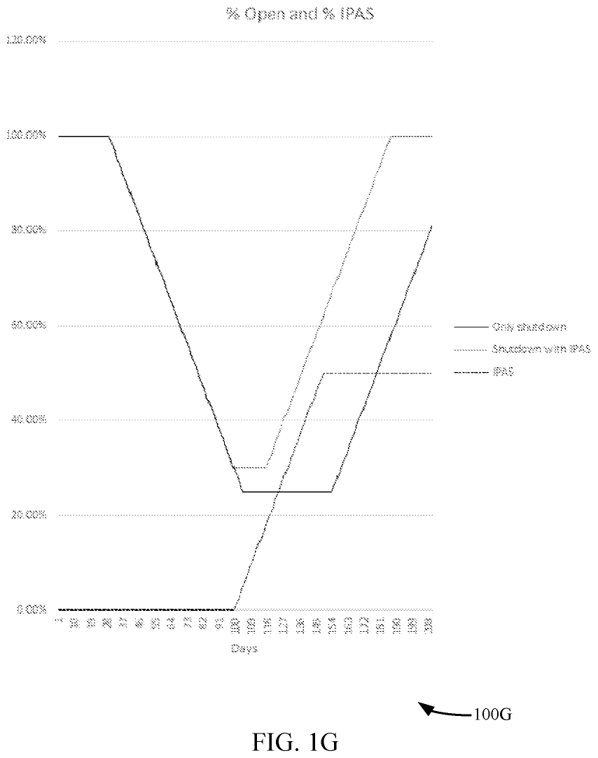

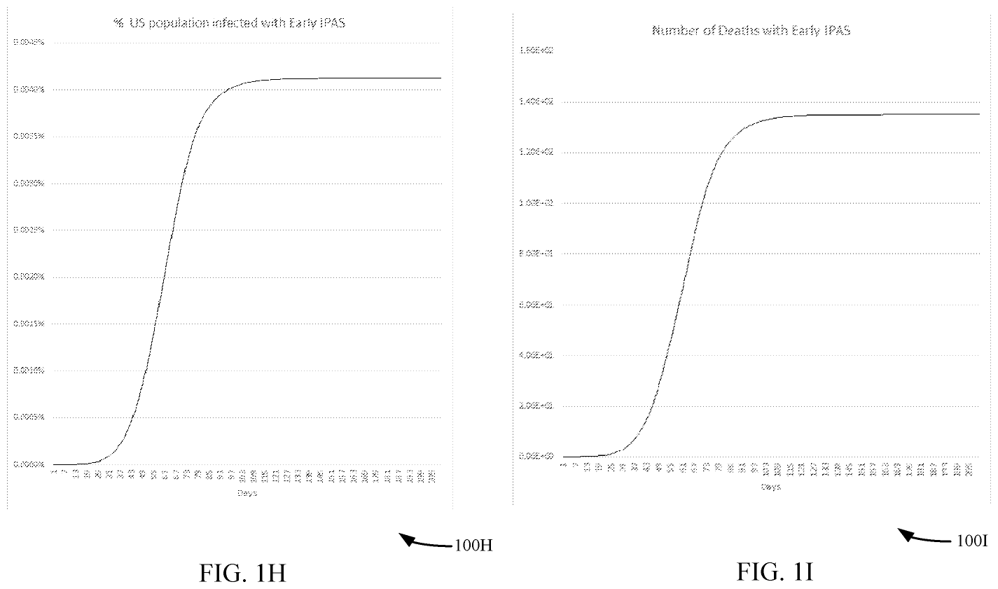

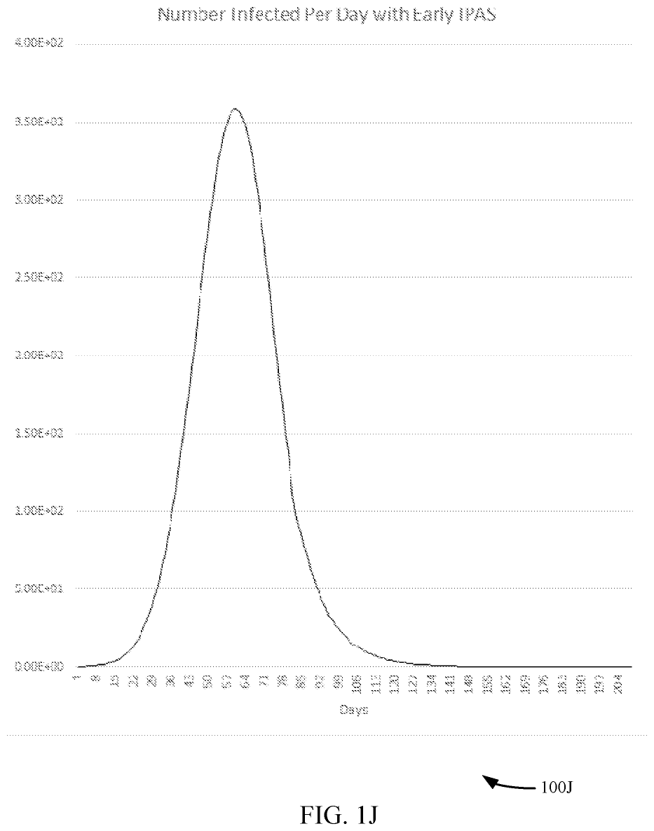

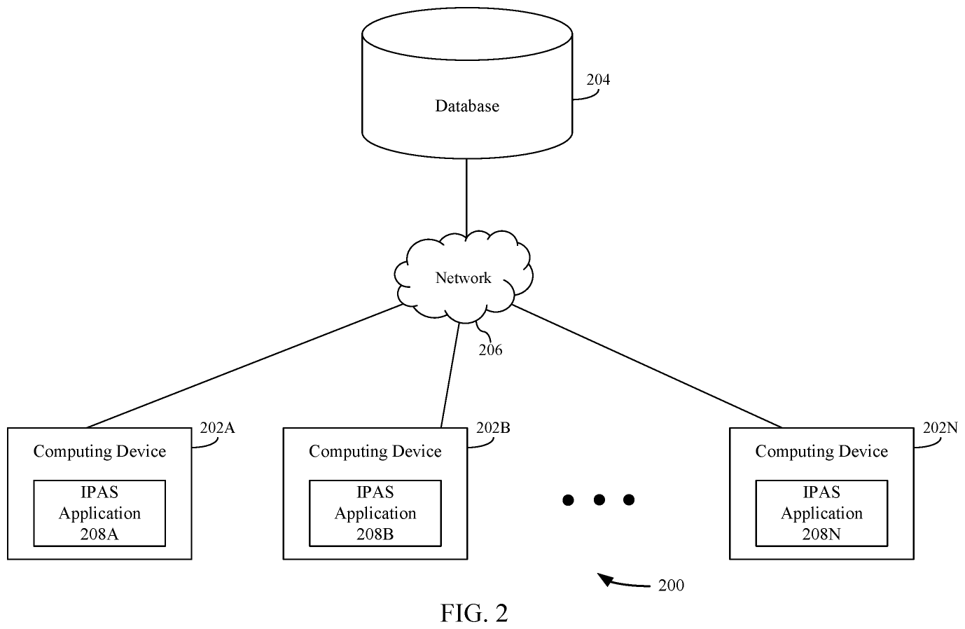

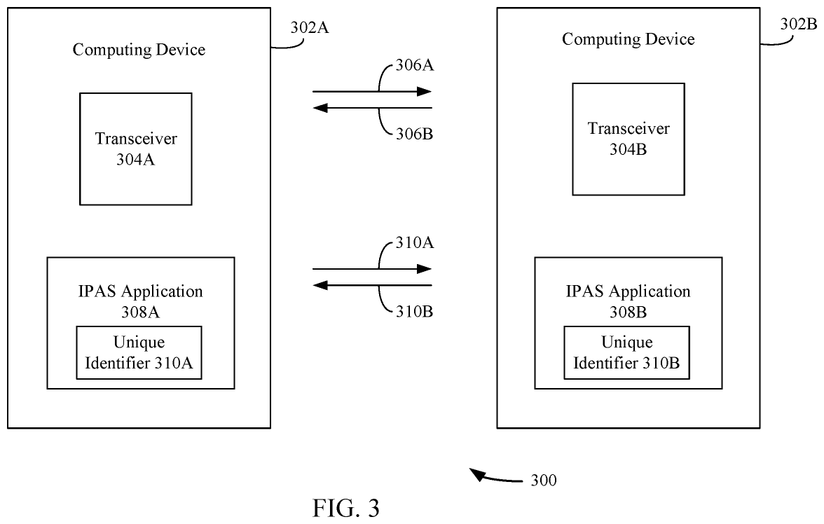

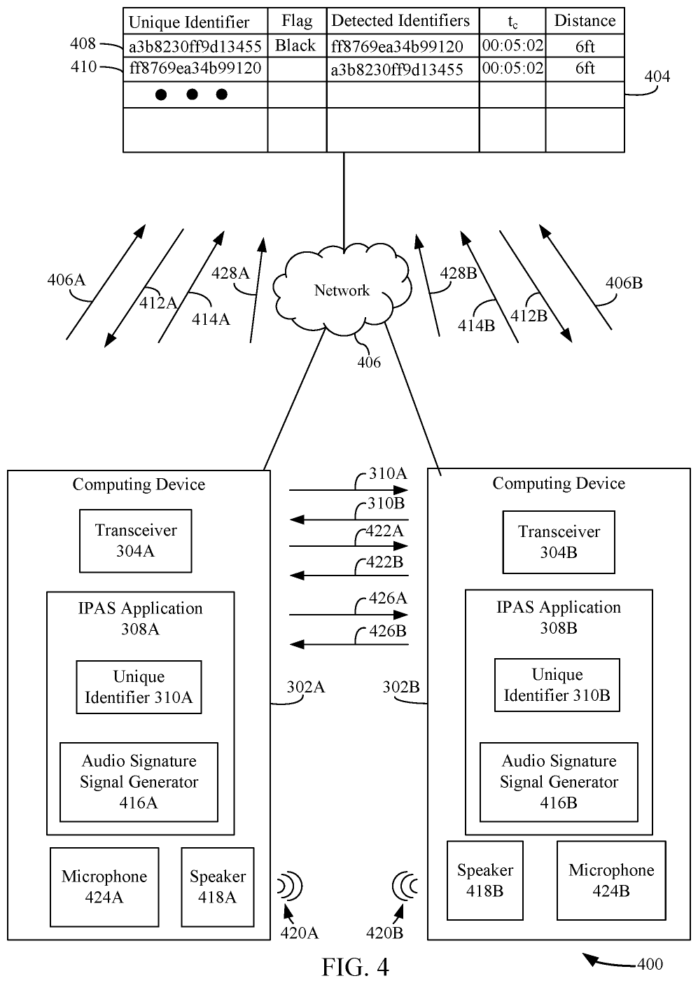

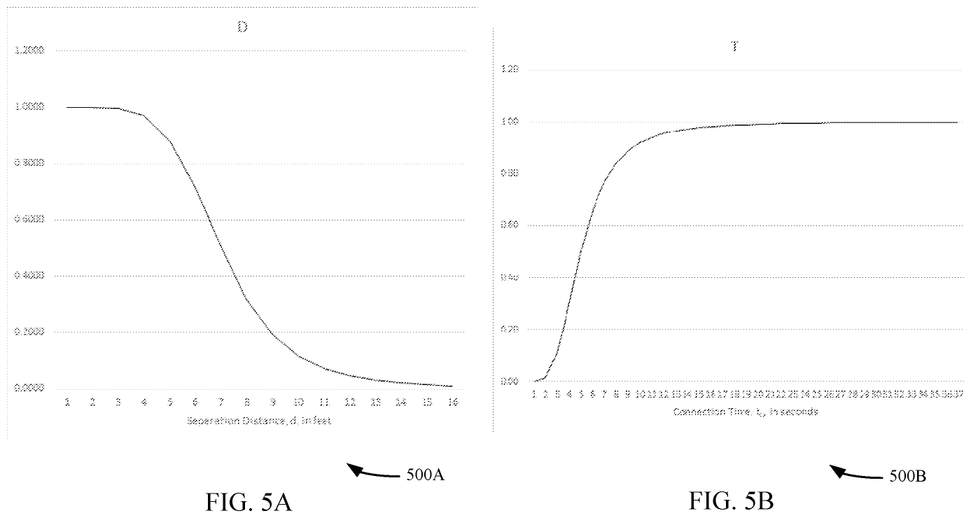

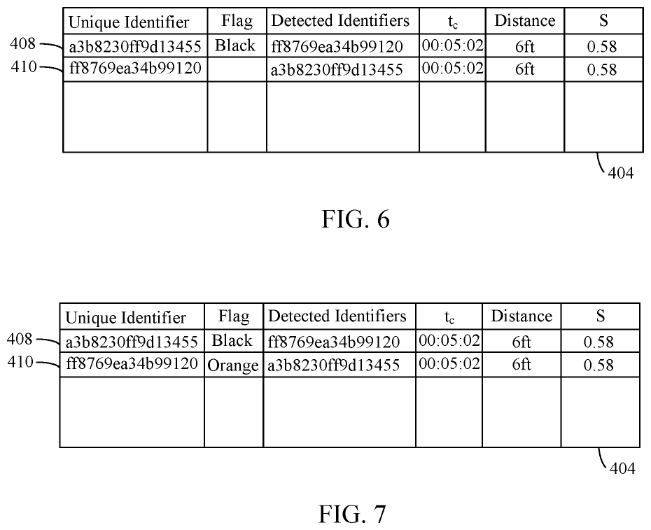

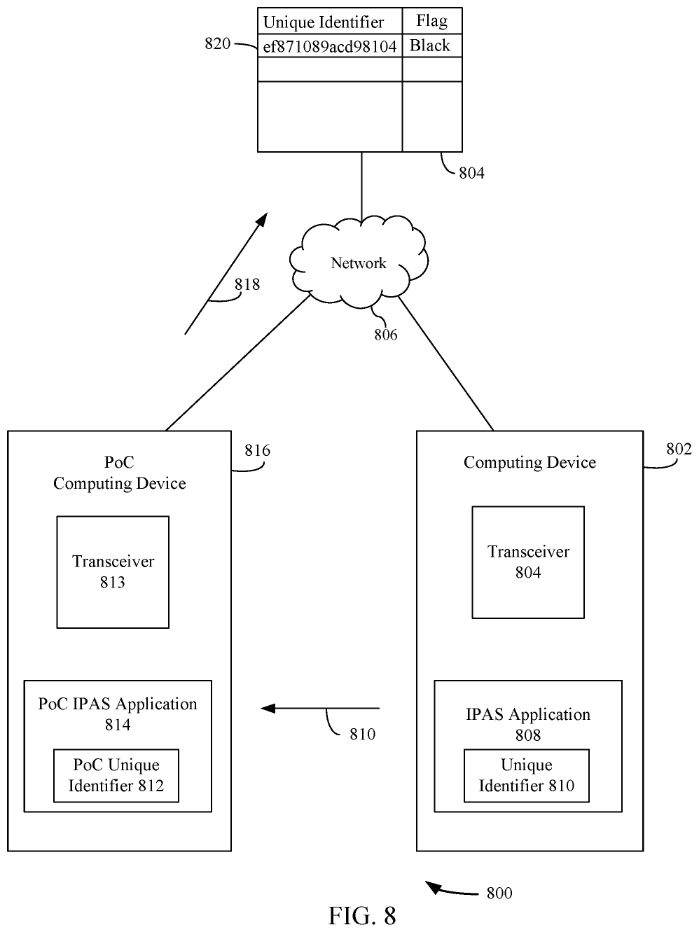

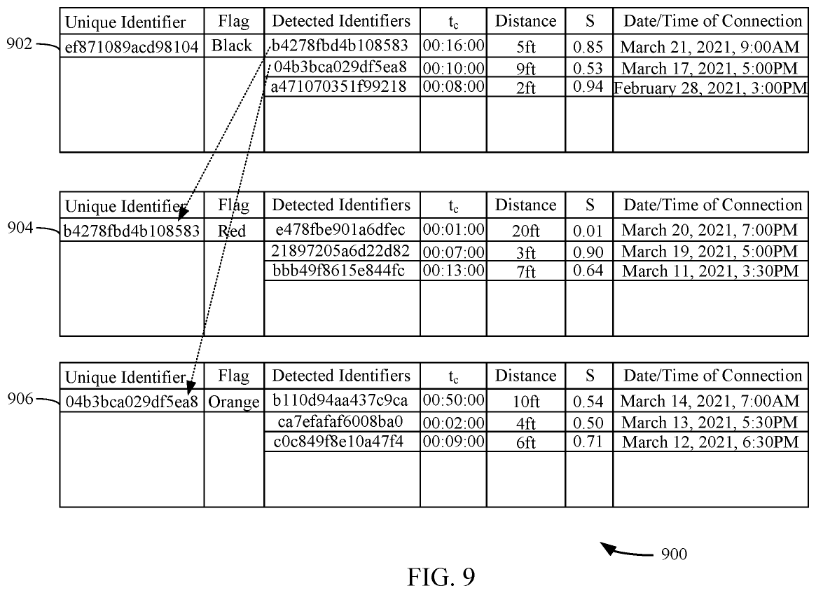

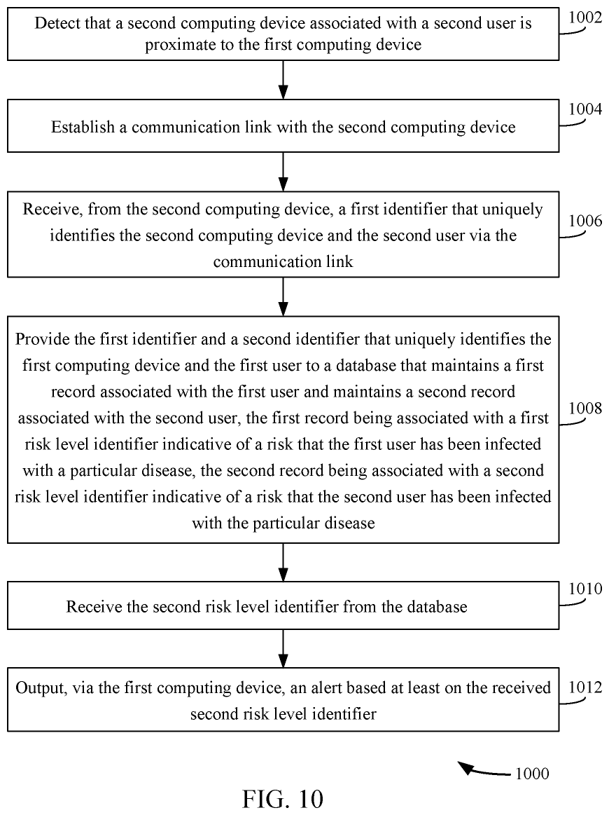

FIG. 1J depicts a graph showing a peak infection rate when deploying an infected person alert system in accordance with embodiments described herein.

FIG. 2 shows a block diagram of an infected person alert system in accordance with an example embodiment.

FIG. 3 shows a block diagram of a system configured to detect the presence of computing devices in accordance with an example embodiment.

FIG. 4 shows a block diagram of a system configured to determine whether a user has or may have been infected with a particular disease in accordance with an example embodiment.

FIG. 5A depicts a graph showing the effect a distance factor on a strength of interaction between users in accordance with an embodiment described herein.

FIG. 5B depicts a graph showing the effect a time factor on a strength of interaction between users in accordance with embodiments described herein.

FIGS. 6-7 depict a plurality of records of a database accordance with an example embodiment.

FIG. 8 depicts a system for updating a risk level identifier at a point-of-care facility in accordance with an example embodiment.

FIG. 9 depicts a plurality of records of a database in accordance with another example embodiment.

FIG. 10 shows a flowchart of a method performed by a first computing device for alerting a first user in accordance with an example embodiment.

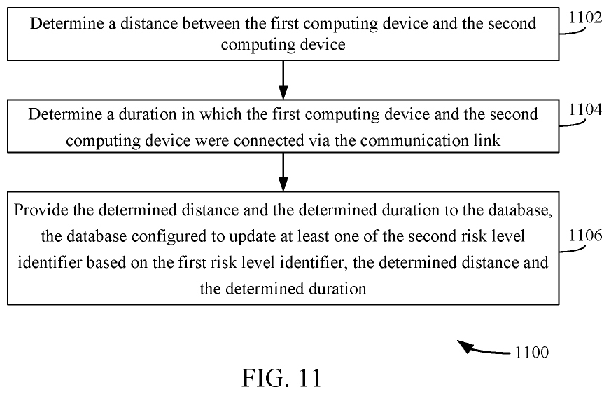

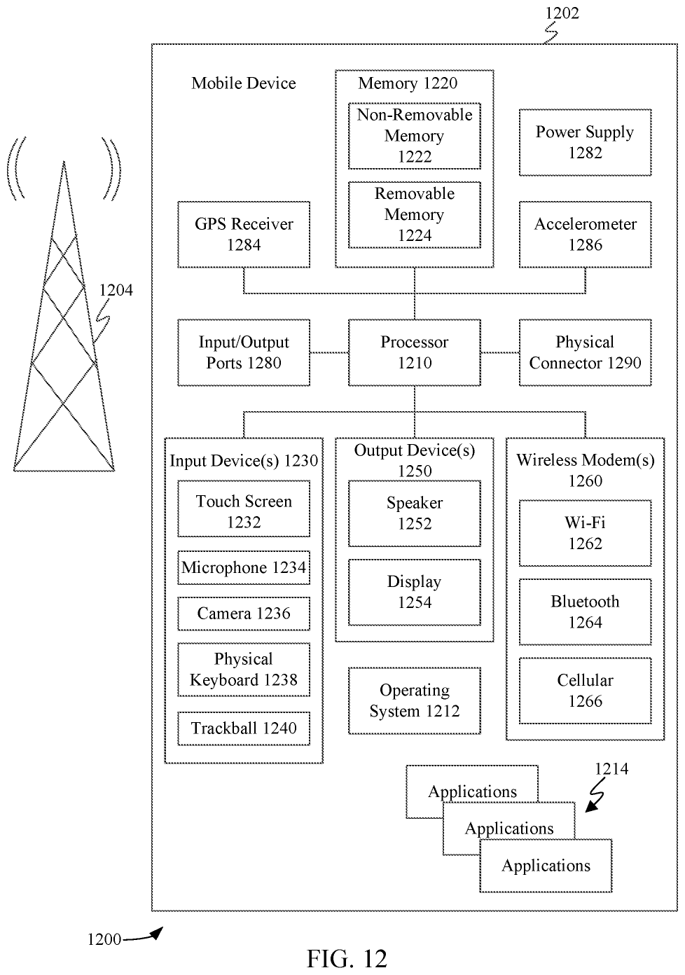

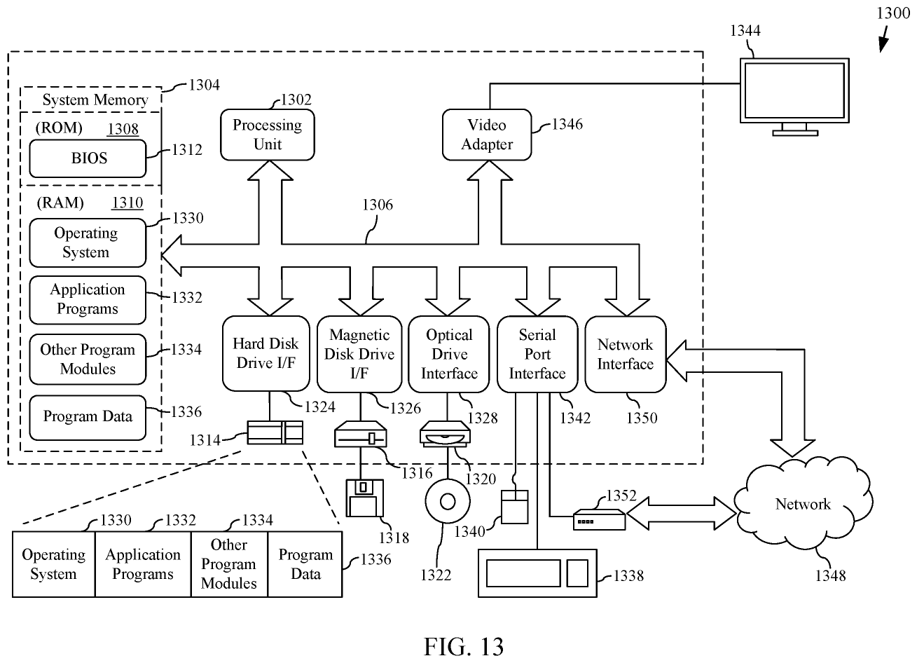

FIG. 11 shows a flowchart of a method for alerting a user based on distance connection duration in accordance with an example embodiment.

FIG. 12 is a block diagram of an exemplary user device in which embodiments may be implemented.

FIG. 13 is a block diagram of an example processor-based computer system that may be used to implement various embodiments.

The features and advantages of the implementations described herein will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION

### I. Introduction

The present specification and accompanying drawings disclose numerous example implementations. The scope of the present application is not limited to the disclosed implementations, but also encompasses combinations of the disclosed implementations, as well as modifications to the disclosed implementations. References in the specification to “one implementation,” “an implementation,” “an example embodiment,” “example implementation,” or the like, indicate that the implementation described may include a particular feature, structure, or characteristic, but every implementation may not necessarily include the particular feature, structure, or characteristic. Moreover, such phrases are not necessarily referring to the same implementation. Further, when a particular feature, structure, or characteristic is described in connection with an implementation, it is submitted that it is within the knowledge of persons skilled in the relevant art(s) to implement such feature, structure, or characteristic in connection with other implementations whether or not explicitly described.

In the discussion, unless otherwise stated, adjectives such as “substantially” and “about” modifying a condition or relationship characteristic of a feature or features of an implementation of the disclosure, should be understood to mean that the condition or characteristic is defined to within tolerances that are acceptable for operation of the implementation for an application for which it is intended.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

Numerous example embodiments are described as follows. It is noted that any section/subsection headings provided herein are not intended to be limiting. Implementations are described throughout this document, and any type of implementation may be included under any section/subsection. Furthermore, implementations disclosed in any section/subsection may be combined with any other implementations described in the same section/subsection and/or a different section/subsection in any manner.

### II. Example Implementations

Epidemics such as the 1918 Spanish Flu and the 2020 coronavirus (i.e., COVID-19) pandemic are driven by the following principle:

$${N}_{i} = {R}_{0} {N}_{c} or \frac{{N}_{i}}{{N}_{c}} = {R}_{0} \qquad ( Equation 1 )$$

where N_{c} is the number of ill and contagious people and R₀ is the number of people who will be infected by one of these people during the infectious period of the illness. Nᵢ then is the total number of people who will be infected during this period. If N_{c} is equal to one, then Nᵢ is equal to R₀. If, for example, R₀ is equal to two and N_{c} is equal to one, then Ni is equal to two. Increasing the number of contagious people to two results in Nᵢ equaling to four, and increasing the number of contagious people to four results in Nᵢ equaling to eight. Accordingly, the number of infected people keeps doubling every contagious period. This is the classical exponential growth curve that drives epidemics. If unchecked, growth is unbounded, as occurred during the 1918 Spanish Flu, which, even with social distancing, ended because those infected either died or developed immunity. For COVID-19, the infection period is considered to be no longer than 10 days after symptom onset. However, the disease progressed rapidly, doubling every 2 to 14 days depending on country and regions within countries, leading to wildly varying values of R₀.

Considering approaches to stop such an epidemic is enabled by adding a term to Equation (1):

$${N}_{i} = {CR}_{0} {N}_{c} or \frac{{N}_{i}}{{N}_{c}} = {CR}_{0} \qquad ( Equation 2 )$$

where C is a contact factor. If contagious persons do not come into contact with others to infect during the infectious period, C will be equal to zero. In accordance with Equation 2, the total number of infected people will also be zero, and the disease will not spread. If C is reduced to and below the value of 1 divided by R₀, the disease cannot spread, as Nᵢ is less than N_{c}, and therefore, will die out over time.

While Equations 1 and 2 are useful to explain the basic concept of infectious growth, the disease does not just increase by the factor CR₀ over the infection period, but rather continuously, every second, every minute, every hour, every day. Assuming a fourteen-day infection period, the daily growth may be modeled in accordance with Equation 3, which is shown below:

$$\frac{{N}_{i}}{{N}_{c}} = \frac{C {R}_{0}}{14} \qquad ( Equation 3 )$$

Equation 3 assumes infected persons no longer contribute to contagion growth fourteen days after infection. Assuming the death rate doubles every three days and assuming a one percent fatality rate from the disease, R₀ is set to 3. However, alternate infection periods and values for R₀ may be utilized depending on the disease being modeled.

Without a vaccine, there are three primary ways to slow or stop epidemic growth by reducing the contact factor C. This may be best understood by separating the contact factor C into three separate components as shown below in Equation 4:

C=C_{q}CₛCₐ, (Equation 4)

C_{q} corresponds to personal behavior to self-quarantine when symptoms become evident. FIGS. 1A-1C depict graphs 100A-100C, which model results for self-quarantine starting day 8 of the 14-day infection period of COVID-19 compared to unabated growth of the disease with respect to the U.S. population. The numbers on the vertical axis for unabated growth show why epidemics are so frightening. As shown in FIGS. 1A-1C, 98% of the U.S. population becomes infected with over 3 million deaths within four months for a 1% fatality rate. As further shown in FIGS. 1A-1C, self-quarantining dramatically reduces the number of people of infected and the number of deaths (by roughly 60%). This is a significant reduction, indicating self-quarantine is an effective tool to be deployed.

Cₛ corresponds to a government mandate for social distancing, which has a huge financial price tag, where people are isolated at home, thereby shutting down the economy. This scenario is shown in graphs 100D-100F of FIGS. 1D-1F with the solid line curve. It is a difficult and painful process to require lock down and isolation mandates. As shown in FIGS. 1D-1F, a complete or partial shutdown (where 25% of the economy remains open) limits the deaths to less than 500,000 with 14% of the population infected. FIG. 1D shows the effect of loosening the shutdown and isolation timeline. The infection and death numbers start ramping up once again. Once shut down, and isolation begins within a country, it will have to remain that way until something happens to keep the value of C low, such as a vaccine.

Cₐ, corresponds to informed contact avoidance with an infected person. There is currently no system in place that warns a person that they may be in proximity with someone who may be infected with a particular disease. The embodiments described herein are directed to such a system. In particular, the embodiments are directed to an infected person alert system (IPAS). For example, the system may comprise computing devices associated with users and a central database. The central database maintains a record for each person enrolled in the IPAS. The record comprises an infection risk level identifier that indicates a risk that the user has been infected with a particular disease. The record is also associated with records associated with other users that were determined to be in close proximity to the user (e.g., within 30 feet). Each computing device enrolled in the IPAS comprises an IPAS application. The IPAS application executing on a first computing device is configured to detect whether a second computing device enrolled in the IPAS is in close proximity therewith. Upon detecting as such, the computing devices exchange identifiers that anonymously and uniquely identify each other. The IPAS application executing on at least one of the computing devices provides the identifier received thereby to the central database. The central database analyzes the record associated with the received identifier (i.e., the detected user's record) and determines the risk level of the user associated with the identifier. The database provides the risk level to the IPAS application, and the IPAS application then provides an alert (e.g., an audible, visual or vibrating alert) on the user's device based on the determined risk level, letting the user know that he is in close proximity with a user that may be infected with the particular disease. The user may then maintain a safe distance from that other person, thereby avoiding contact with that person and preventing the disease from spreading. The central database may also update the risk level associated with a particular record based on the risk levels of the other records associated therewith. For example, if a particular record has a risk level that indicates that the user has a particular disease, records associated therewith may also be updated with a new risk level. The new risk level may be dependent on the strength of interactions between the users associated with such records (e.g., the time the users spent with each other, the distance between the users between interactions, etc.). The IPAS application associated with a particular user may periodically communicate with the central database to determine the user's risk level and alert the user accordingly.

Such functionality is achieved efficiently due to the arrangement of the components that comprise the IPAS. For example, the central database is configured to receive risk level identifiers from computing devices enrolled in the IPAS, and link users of the computing devices based on their proximity to each other (based on information provided by the computing devices). If a risk level identifier indicates that a particular user is infected with a disease, the database determines the users that were proximate to that user and updates their risk level identifiers to indicate that they now may be infected. The IPAS applications executing on the computing devices periodically communicate with the central database to determine the users' risk levels and alert the users accordingly. The users then takes the appropriate action to prevent the spread of the disease.

Accordingly, the embodiments described herein enable the reduction of each of the contact factor components C_{q}, Cₛ, and Cₐ, and therefore, advantageously prevents contact with infected people, takes infected people out of circulation, and is capable stopping both an emerging epidemic and one in full progress after other initiatives have been started, such as shutdown and isolation.

The foregoing techniques enable contact avoidance and alert notifications to be performed without compromising the privacy of the users enrolled in the IPAS. For example, the records of the database are stored in anonymous fashion, where each record is identified by a globally-unique identifier. There is no personal information (names, addresses, phone numbers, location information, etc.) maintained by the database. Accordingly, the privacy of the users enrolled in the IPAS is protected from both governments agencies that may implement the IPAS or malicious entities (e.g., hackers) seeking to exploit the data in the database.

The results of applying the IPAS with respect to the second contact factor Cs are shown via the dotted lines shown in FIGS. 1D-1F. A major impact of getting the IPAS operational for this case is stopping the epidemic, thus allowing the safe, full 100% re-opening of a country from the shutdown as shown in FIGS. 1D-1F.

FIG. 1G depicts a graph 100G which shows the IPAS ramping up to 50% effectiveness (also enabling a net 50% effectiveness in self-quarantine) and remaining there. As shown in FIG. 1G, when shutdown is ramped down where only a net average of 25% of the US is open, the growth of the epidemic is stopped, as shown in FIG. 1F. The IPAS keeps it stopped, allowing the US to open back up to 100% operation (as shown by the dotted line in FIG. 1G). In FIG. 1G, the solid line shows the attempted ramp up to 80% open without the IPAS in place, which allows the epidemic to roar back with exponential growth as shown in FIG. 1F via the solid line. Once shut down to even past the peak of infection and death rate, the shutdown will have to remain in place until the IPAS is deployed to keep the pandemic controlled, as the shutdown is relaxed and totally removed.

FIGS. 1H-1J depicts graphs 100H-100J showing the effect of having the IPAS operational at the beginning of a novel virus outbreak. As shown in FIGS. 1H-1J, the IPAS quickly quenches the outbreak to prevent it from becoming an epidemic. For COVID-19, only 63,000 people would become infected with 630 deaths. This scenario demonstrates that the IPAS can prevent epidemics of any novel contagion such as what occurred with the Spanish Flu and COVID-19, thereby saving hundreds of thousands of lives and countless trillions of dollars of shut down economic loss.

FIG. 2 shows a block diagram of an infected person alert system (IPAS) 200 in accordance with an example embodiment. As shown in FIG. 2, system 200 includes computing devices 202A-202N and a central database 204 that are communicatively coupled via a network 206. Each of computing devices 202A-202N may store and execute an IPAS application (e.g., IPAS applications 208A, 208B, and 208N). As will be described herein, each of IPAS applications 208A, 208B, and 208N is configured to alert a user when the user is in the presence of (e.g., proximately located to) another user that is associated with particular risk level with respect to a particular disease. Examples of computing devices 202A-202N include, but are not limited to, a smart phone, a tablet, a cell phone, a personal data assistant, a desktop or laptop computer, etc. In accordance with an embodiment, one or more of computing devices 202A-202N may be a standalone (e.g., custom) device that is specifically configured to execute IPAS applications 208A-208N, respectively, and provide alerts to users. Network 206 may comprise one or more networks such as local area networks (LANs), wide area networks (WANs), enterprise networks, the Internet, etc., and may include one or more of wired and/or wireless portions.

IPAS applications 208A-208N and database 204 may be a national or global resource and would ideally be used by a government agency (e.g., the Center of Disease Control (CDC), the World Health Organization (WHO), etc.). IPAS applications 208A-208N may be pre-loaded on respective computing devices 202A-202N, or alternatively, may be downloaded thereto (e.g., via a software update, via a website maintained by a government agency, etc.).

With the arrival of a new pathogen, such as COVID-19, with lethality warranting a national or world health organization response, countries may mandate that their citizens download IPAS applications 208A-208N onto their respective devices (e.g., computing devices 202A-202N). Each of IPAS applications 208A-208N are provided a unique identifier. That is, each IPAS application that is provisioned to a computing device is assigned a different, unique identifier. For example, the website from which IPAS applications 208A-208N are downloaded may comprise a random number generator. The random number generator may generate a random number and assign the random number to an IPAS application that is downloaded by a user. The random number generator ensures that a particular random number is generated only one time, thereby preventing more than one IPAS application from being assigned the same random number.

The identifier may be a 32-bit random number, a 64-bit random number, etc., where each of the random numbers are only utilized once globally (i.e., worldwide). The number of bits utilized for the random number may depend on the number of IPAS applications that are expected to be downloaded. For instance, a 32-bit random number provides roughly 4.3 billion unique numbers. If the number of IPAS applications anticipated to be downloaded is less than or equal to this number, than a 32-bit number may suffice. However, if a greater number of IPAS applications are anticipated to be downloaded, then a 64-bit or 128-bit number, for example, may be utilized.

Upon download, each of IPAS applications 208A-208N may prompt the user to enter authentication information that is utilized to authenticate the user to utilize the IPAS application installed on his or her computing device. Examples of authentication information include, but are not limited to, a username and/or password, a personal identification number (PIN), biometric information (e.g., a voice sample, a facial scan, a fingerprint scan, etc.). When utilizing the IPAS application, the user will be required to enter provide valid authentication information in order to utilize the IPAS application.

For each IPAS application provisioned, a record may be created in database 204. The record is initially associated with the unique identifier assigned to the IPAS application. The record is also associated with a risk level identifier indicative of a risk that the user associated with the record has been infected with a particular disease. Initially, the flag may be set to a default value that indicates that the user does not have the disease or was not in proximity with another user that may have the disease. In accordance with an embodiment, the record is generated after the IPAS application is downloaded to a particular computing device of computing devices 202A-202N. In accordance with such an embodiment, the IPAS application provides a request to add a record to database 204. The request may comprise the unique identifier assigned to the IPAS application. For example, after IPAS application 208A is downloaded onto computing device 202A, IPAS application 208A may send a request to database 206 to add a record corresponding thereto. After IPAS application 208B is downloaded onto computing device 202B, IPAS application 208B may send a request to database 206 to add a record corresponding thereto. After IPAS application 208N is downloaded onto computing device 202N, IPAS application 208N may send a request to database 206 to add a record corresponding thereto. In accordance with another embodiment, the website that provisions IPAS applications 208A-208N may provide a request to database 206 to add such records. For example, when the website receives a request to download an IPAS application, the website may provide the IPAS application to the computing device that submitted the request. After provisioning the IPAS application to the computing device, the website provides a request comprising the unique identifier assigned to the provisioned IPAS application to database 206.

Each of IPAS applications 208A-208N are configured to detect the presence of other computing devices that are proximate thereto. For example, FIG. 3 shows a block diagram of system 300 configured to detect the presence of computing devices in accordance with an example embodiment. As shown in FIG. 3, system 300 comprises a first computing device 302A and a second computing device 302B, which are examples of computing devices 202A-202N, as described above with reference to FIG. 2. Computing device 302A comprises an IPAS application 308A and a transceiver 304A. Computing device 302B comprises an IPAS application 308B and a transceiver 304B. IPAS applications 308A and 308B are examples of IPAS applications 208A-208N, as described above with reference to FIG. 2. IPAS application 308A is associated with a unique identifier 310A, which may be assigned to IPAS application 308A at the time IPAS application 308A is provisioned or downloaded to computing device 302A. IPAS application 308B is associated with a unique identifier 310B, which may be assigned to IPAS application 308B at the time IPAS application 308B is provisioned or downloaded to computing device 302B.

Each of transceivers 304A and 304B may comprise one or more antennas that are configured to transmit and receive radio frequency (RF) signals to and from other devices. Transceivers 304A and 304B may be configured to transmit and receive RF signals in accordance with one or more protocols/standards. For example, transceivers 304A and 304B may be configured to transmit and receive RF signals in accordance with certain RF-based short-range communication technologies such as Bluetooth™ or Bluetooth™ Low Energy (BLE) as described in the various standards developed and licensed by the Bluetooth™ Special Interest Group, or technologies such as ZigBee® that are based on the IEEE 802.15.4 standard for wireless personal area networks (specifications describing ZigBee are publicly available from the ZigBee® Alliance). In another example, transceivers 304A and 304B may be configured to transmit RF signals in accordance with one or more cellular standards, such as Code Division Multiple Access (CDMA), Time Division Multiple Access (TDMA), Frequency Division Multiple Access (FDMA), Frequency Division Duplex (FDD), Global System for Mobile Communications (GSM), Wideband-CDMA (W-CDMA), Time Division Synchronous CDMA (TD-SCDMA), Long-Term Evolution (LTE), Time-Division Duplex LTE (TDD-LTE) system, and/or the like. In yet another example, transceivers 304A and 304B may be configured to transmit RF signals in accordance with other RF-based communication technologies such as any of the well-known IEEE 802.11 protocols. It is noted that transceivers 304A and 304B may include various components (e.g., one or more antennas) that are not shown in FIG. 3 for the sake of brevity. It is further noted that rather than using transceivers 304A and 304B, each of computing devices 302A and 302B may utilize separate transmitters and receivers for transmitting and receiving RF signals, respectively.

In accordance with an embodiment, each of transceivers 304A and 304B are configured to periodically broadcast a beacon signal that is detectable by other computing devices configured to detect the beacon signal and that are within range of transmission of the beacon signal. For example, transceiver 304A may periodically transmit a beacon signal 306A, and transceiver 304B may periodically transmit a beacon signal 306B. Computing devices (e.g., computing devices 302A and 302B) that are in range of transmission of a beacon signal such that the beacon signal may be detected/received are considered to be proximate to the computing device that transmits the beacon signal. Accordingly, whether devices are considered proximate may be dependent on the type of wireless communication protocol utilized to transmit beacon signals, as different wireless communication protocols support different ranges. In an embodiment in which beacon signals 306A and 306B are transmitted in accordance with a Class 1 Bluetooth™ or BLE protocol, the range of transmission of beacon signals 306A and 306B can reach approximately 100 meters (300 feet). In accordance with such an embodiment, computing devices 302A and 302B can be determined to be proximate if they are within approximately 100 meters (i.e., in a range in which they can detect the Class 1 Bluetooth™ beacon signals). In an embodiment in which beacon signals 306A and 306B are transmitted in accordance with a Class 2 Bluetooth™ or BLE protocol, the range of transmission of beacon signals 306A and 306B can reach approximately 10 meters (33 feet). In accordance with such an embodiment, computing devices 302A and 302B can be determined to be proximate if they are within approximately 10 meters (i.e., in a range in which they can detect the Class 2 Bluetooth™ beacon signals). In an embodiment in which beacon signals 306A and 306B are transmitted in accordance with a Class 3 Bluetooth™ or BLE protocol, the range of transmission of beacon signals 306A and 306B can reach 1 meter (3 feet). In accordance with such an embodiment, computing devices 302A and 302B can be determined to be proximate if they are within approximately 1 meter (i.e., in a range in which they can detect the Class 3 Bluetooth™ beacon signals). Each of transceivers 304A and 304B are configured to receive and detect beacon signals 306A and 306B, respectively. Note that Bluetooth™ is disclosed herein as an example short-range wireless communication standard applicable to embodiments. In further embodiments, other types of short-range wireless communication standards may be implemented to transmit and receive beacon signals.

Detection of beacon signal 306B may trigger computing device 302A to perform one or more actions. Similarly, detection of beacon signal 306A may trigger computing device 302B to perform one or more actions. For instance, computing device 302A and computing device 302B may establish a peer-to-peer network or a personal area network (i.e., communication link) by which computing devices 302A and 302B communicate information. After establishing the link, IPAS application 308A may be configured to transmit unique identifier 310A to computing device 302B via transceiver 304A and the communication link. Similarly, IPAS application 308B may be configured to transmit unique identifier 310B to computing device 302A via transceiver 304B and the communication link.

After receiving unique identifier 310B, IPAS application 308A communicates with a database (e.g., database 204) to determine whether the user associated with unique identifier 310B has been or may have been infected with a particular disease. Similarly, after receiving a unique identifier 310A, IPAS application 308B communicates with the database (e.g., database 204) to determine whether the user associated with unique identifier 310A has been or may have been infected with the particular disease.

For example, FIG. 4 shows a block diagram of system 400 configured to determine whether a user has or may have been infected with a particular disease in accordance with an example embodiment. As shown in FIG. 4, system 400 comprises computing devices 302A and 302B and a database 404, which is an example of database 204, as shown in FIG. 2. As shown in FIG. 4, database 404 is represented as a database table comprising a plurality of records 408 and 410, database 404 may further comprise one or more applications, functions and/or stored procedures that are configured to perform various operations with respect to the data and/or records maintained thereby. Each of computing devices 302A and 302B and database 404 are communicatively coupled via a network 406, which is an example of network 206, as described above with reference to FIG. 2.

Responsive to detecting beacon signal 310B, IPAS application 308A provides a request 406A to database 404 via network 406. Request 406A comprises unique identifier 310A and unique identifier 310B. Request 406A may also comprise additional information, including, but not limited to, a date and/or time at which computing device 302A established communication with computing device 302B. Responsive to detecting beacon signal 310A, IPAS application 308B provides a request 406B to database 404 via network 406. Request 406B comprises unique identifier 310A and unique identifier 310B. Request 406B may also comprise additional information, including, but not limited to, a date and/or time at which computing device 302B established communication with computing device 302A.

Database 404 stores a record for each user enrolled into the IPAS system. The only identifying information stored by database 404 is the unique identifiers of the IPAS applications installed by such users. Accordingly, the records are stored in an anonymous fashion, thereby protecting the privacy of the users. In the example shown in FIG. 4, database 404 stores two records 408 and 410, although database 404 may maintain any number of records (e.g., millions or billions of records). Record 408 comprises a unique identifier field that stores unique identifier 310A for IPAS application 308A (e.g., “a3b8230ff9d13455”) and a risk level identifier field that stores the risk level identifier for the user of IPAS application 308A. Record 410 comprises a unique identifier field that stores unique identifier 310B for IPAS application 308B (e.g., “ff8769ea34b99120”) and a risk level identifier field that stores the risk level identifier for the user of IPAS application 308B. Unique identifiers 310A and 310B are shown as being 64-bit numbers. However, the embodiments described herein are not so limited. Each of records 408 and 410 also comprises a detected identifiers field that stores unique identifiers associated with other users that were detected to be proximate to the computing device associated with the unique identifier stored in the unique identifier field. Each of records 408 and 410 may comprise additional information, e.g., the time and/or date at which communication was established with another user. Further information that may be stored by records maintained by database 404 is further described below. It is noted that while database 404 is represented as a database table comprising a plurality of records 408 and 410, the embodiments described herein are not so limited. For example, database 404 may comprise a folder representative of each record. The folder may comprise files and/or folders representative of the detected identifiers. Each of the folders may be associated with metadata corresponding to one or more of the database table field described herein.

Responsive to receiving request 406A, database 404 is configured to associate unique identifier 310B with unique identifier 310A, as a user associated with unique identifier 310B has been detected to be proximately located to the user associated with unique identifier 310A. Accordingly, as shown in FIG. 4, database 404 has populated the detected identifier field associated with unique identifier 310A to include unique identifier 310B. Database 404 is also configured to determine and/or store a risk level identifier associated with unique identifier 310B. In the example shown in FIG. 4, no risk level is associated with unique identifier 310B. Database 404 returns a response 412A indicating that no risk level identifier is associated with computing device 302A via network 406. Transceiver 304A receives response 412A, and response 412A is provided to IPAS application 308A. IPAS application 308A analyzes response 412A to determine the risk level identifier included therein. IPAS application 308A may alert the user via computing device 302A if the risk level identifier is indicative of a risk that the user has been or may have been infected with a particular disease. In the example described above, no such alert is issued, as there is no risk level identifier associated with the other user. However, in certain embodiments, an alert may be issued that indicates that the status of the user is not known, and that the user should take precaution.

Responsive to receiving request 406B, database 404 is configured to associate unique identifier 310A with unique identifier 310B, as a user associated with unique identifier 310A has been detected to be proximately located to the user associated with unique identifier 310B. Accordingly, as shown in FIG. 4, database 404 has populated the detected identifier field associated with unique identifier 310B to include unique identifier 310A. Database 404 is also configured to determine a risk level identifier associated with unique identifier 310A. In the example shown in FIG. 4, the risk level identifier associated with unique identifier 310B is “Black.” A risk level identifier of “Black” may indicate that the user associated with unique identifier 310A has been infected with the disease. Database 404 returns a response 412A comprising the risk level identifier to computing device 302B via network 406. Transceiver 304B receives response 412B, and response 412B provided to IPAS application 308B. IPAS application 308B analyzes response 412B to determine the risk level identifier included therein. In this example, IPAS application 308B alerts the user via computing device 302B because the risk level of the user associated with unique identifier 310 is “Black.” Additional details regarding the types of alerts that IPAS applications 308A and 308 may issue and the various risk levels are described below.

Each of IPAS applications 308A and 308B are configured to determine a duration of time in which computing devices 302A and 302B are connected (referred herein as a connection time t_{c}). Such information may be utilized to determine a length of time in which users were in close proximity. For example, upon establishing a communication link by which computing devices 302A and 302B communicate, each of IPAS applications 308A and 308 may initiate a timer. The timer is stopped after the connection or network between computing devices 302A and 302B is terminated (e.g., when computing devices 302A and 302B are no longer in range). Each of IPAS applications 308A and 308B provide its determined connection time to database 404 via network 406. For example, as shown in FIG. 4, IPAS application 308A provides a request 414A comprising unique identifier 310A and the determined connection time to database 404 via network 406, and IPAS application 308B provides a request 414B comprising unique identifier 310B and the determined connection time to database 404 via network 406. Database 404 stores the received connections times in a connection time field (t_{c}) of database 404. For example, as shown in FIG. 4, database 404 stores the connection time received via request 414A in the connection time field of record 408 (as identified by unique identifier 310A included in request 414A) and stores the connection time received via request 414B in the connection time field of record 410 (as identified by unique identifier 310B included in request 414B). In this example, both IPAS applications 308A and 308B have determined a similar connection time (5 minutes and 2 seconds) with respect to each other. Database 404 associates the connection time received via request 414A with the unique identifier specified in the detected identifier field of record 408 and associates the connection time received via request 414B with the unique identifier specified in the detected identifier field of record 410.

It has been observed that certain diseases are generally more transmissible if people are within close proximity for a certain length of time. For example, for the coronavirus, it has been observed that a person is more likely to be exposed to the coronavirus if they are in close proximity (e.g., 6 feet) for periods lasting at least 15 minutes with someone that is infected with the coronavirus. In accordance with an embodiment, to reduce the power consumption of computing devices 302A and 302B, IPAS applications 308A and 308B are configured to stop their respective timers after expiration of a predetermined period of time (e.g., 15 minutes).

Each of IPAS applications 308A and 308B and/or database 404 may be configured to determine the distance between computing devices 302A and 302B. Both Global Positioning System (GPS) and cell tower-based phone location capabilities are generally poor and may not be relied upon to provide accurate data representative of devices being within feet of each other. A possible viable alternative would be to use audio signal timing. It is likely that such a signal may be faint because of distance and/or path blockage (such as the device (e.g., a smart phone) being in a pocket, purse, or backpack, for example) and will have to compete in a noisy environment. To overcome such issues, the embodiments described herein utilize an audio signature signal.

For example, each of IPAS applications 308A and 308B may comprise an audio signature signal generator 416A and 416B. Audio signature signal generator 416A is configured to generate an audio signature signal 420A that is unique to computing device 302A. For instance, audio signature signal generator 416A may utilize communication parameters that are specific to computing device 302A to generate and/or transmit audio signature signal 420A via speaker 418A of computing device 302A. The communication parameters may be based on unique identifier 310A. For instance, audio signature signal generator 416A may comprise circuitry, logic, etc., that receives unique identifier 310A as an input and outputs the communication parameters based on unique identifier 310A. In accordance with the embodiment, the communication parameters comprise a frequency at which audio signature signal 420A is transmitted, an audio pattern to be utilized to transmit audio signature signal 420A, etc.

Similarly, audio signature signal generator 416B is configured to generate an audio signature signal 420B that is unique to computing device 302B. For instance, audio signature signal generator 416B may utilize communication parameters that are specific to computing device 302B to generate and/or transmit audio signature signal 420B via speaker 418B of computing device 302B. The communication parameters may be based on unique identifier 310B. For instance, audio signature signal generator 416B may comprise circuitry, logic, etc., that receives unique identifier 310B as an input and outputs the communication parameters based on unique identifier 310B. Accordingly, each computing device enrolled in the IPAS system may generate a different type of audio signature signal.

The audio signature signal is meant to be detected and processed only by the computing device determined to be proximate to the computing device transmitting the audio signature signal and by which a communication link has been established via beacon signals 310A or 310B (e.g., a peer-to-peer network or a personal area network). To ensure that only computing device 302B detects and processes audio signature signal 420A, IPAS application 308A may transmit the communication parameters determined by audio signature signal generator 416A to computing device 302B via a signal 422A transmitted by transceiver 304A and the communication link established therebetween. Similarly, IPAS application 308B may transmit the communication parameters determined by audio signature signal generator 416B to computing device 302A via a signal 422B transmitted by transceiver 304B and the communication link established therebetween.

Responsive to receiving signal 422A, IPAS application 308B configures itself to detect audio signature signal 420A in accordance with the received communication parameters (i.e., IPAS application 308B effectively creates a filter by which to detect audio signature signal 420A). For instance, IPAS application 308B may configure microphone 424B so that it only detects audio signals in a particular frequency range specified by the parameters, or IPAS application 308B may configure itself to only process audio signals in a particular frequency range or having a particular pattern specified by the parameters. Similarly, IPAS application 308A configures itself to detect audio signature signal 420B in accordance with received communication parameters (i.e., IPAS application 308A effectively creates a filter by which to detect audio signature signal 420B). For instance, IPAS application 308A may configure microphone 424A so that it only detects audio signals in a particular frequency range specified by the parameters, or IPAS application 308A may configure itself to only process audio signals in a particular frequency range or having a particular pattern specified by the parameters. Such a technique is especially effective in crowded environments where multiple signature audio signals are being transmitted from multiple computing devices.

To determine the distance between computing devices 302A and 302B, each of IPAS applications 308A and 308B initiate a timer at the time their respective audio signature signals are transmitted. When IPAS application 308B detects audio signature signal 420A, IPAS application 308B transmits a response signal 426B to computing device 302A via transceiver 304B and the communication link established therebetween. Responsive to receiving response signal 426B, IPAS application 308A may stop the timer to record the time t_{d}, which represents the time it took audio signature signal 420A to travel from computing device 302A to computing device 302B. When IPAS application 308A detects audio signature signal 420B, IPAS application 308A transmits a response signal 426A to computing device 302B via transceiver 304A and the communication link established therebetween. Responsive to receiving response signal 426A, IPAS application 308B may stop the timer to record the time t_{d}, which represents the time it took audio signature signal 420A to travel from computing device 302A to computing device 302B.

Alternatively, IPAS application 308A may maintain a time value (e.g., a timestamp) at which audio signature signal 420A was transmitted and response 422A may comprise a time value at which audio signature signal 420A was received by computing device 302B. IPAS application 308A may determine time to by subtracting the two time values. Similarly, IPAS application 308B may maintain a time value (e.g., a timestamp) at which audio signature signal 420B was transmitted and response 422B may comprise a time value at which audio signature signal 420B was received by computing device 302A. IPAS application 308B may determine time to by subtracting the two time values.

In accordance with an embodiment, IPAS applications 308A and 308 provide the determined time to t_{d} database 404 via requests 428A and 428B, respectively, that are transmitted via network 406. Each of requests 428A and 428B may also comprise unique identifies 310A and 310B. Database 404 determines the distance between computing devices 302A and 302B based on t_{d}. For instance, database 404 may determine the distance in accordance with Equation 5, which is shown below:

Distance=t_{d}vₛ (Equation 5)

where vₛ is equal to 1,125 feet/second (i.e., the velocity of sound). Database 404 stores the determined distance between computing devices 302A and 302B in a distance field of the records associated with the unique identifiers (as specified by requests 428A and 428B) of computing devices 302A and 302B. For example, as shown in FIG. 4, database 404 stores the determined distance (i.e., 6 feet) in the distance field of both records 408 and 410.

In accordance with an embodiment, each of IPAS applications 308A and 308B may determine the distance in accordance with Equation 5 and include the determined distance in requests 428A and 428B, respectively.

In accordance with an embodiment, rather than having both IPAS applications 308A and 308B determine respective t_{d}, only one of IPAS applications 308A and 308B may determine t_{d}. The IPAS application that determines t_{d} may provide the determined t_{d} to the other IPAS application. It is noted that when determining t_{d} any known non-negligible electronic data processing time may be subtracted out to improve accuracy.

In accordance with an embodiment, IPAS applications 308A and 308B may periodically perform the foregoing process to determine the distance between computing devices 302A and 302B. This way, the distance between computing devices 302A and 302B may be updated as the users move around in their environment.

Computing devices 302A and 302B and/or database 404 may be configured to determine an interaction (or connection) strength factor between two users. The interaction factor may be utilized to set risk level identifiers in database 404 for different users. The interaction factor may be based on a distance factor and a time factor. The distance factor D may be determined in accordance with Equation 6, which is shown below:

D=1/(1+(d/d₀)ⁿ) (Equation 6)

where n and d₀ are calibration factors specified by a government agency, such as the CDC or WHO, and d represents the distances calculated via Equation 5. Equation 6 demonstrates that when the determined distance is zero, the distance factor is one, when the determined distance is equal to do, the distance factor is equal to one half, and when the determined distance is large, the distance factor tends to zero. An example curve demonstrating how the distance factor changes is shown in FIG. 5A, where n is set to five, and d₀ is set to 6 feet (i.e., a distance considered to be safe from transmission).

The time factor T may be determined in accordance with Equation 7, which is shown below:

$$T = ( {( \frac{{t}_{c}}{{t}_{0}} )}^{m} ) / ( 1 + {( {t}_{c} / {t}_{0} )}^{m} ) \qquad ( Equation 7 )$$

where m and to are calibration factors specified by a government agency. Equation 7 demonstrates that when tris relatively small (e.g., a few seconds), the time factor T is small, and when t_{c} is relatively large, the time factor T tends to the value of one. An example curve demonstrating how the time factor changes is shown in FIG. 5B, where to is set to four seconds, and m is set to three.

The interaction strength factor S may be determined in accordance with Equation 8, which is shown below:

S=(D+T)/2 (Equation 8)

In accordance with Equation 8, the interaction strength factor S has a maximum value of one, when distance factor D is a relatively short distance (e.g., less than four feet), and time factor T is relatively long (e.g., longer than eight seconds).

Equations 6-8 accommodate various time and distance scenarios. For example, when the distance between users is relatively short and the time in which the users are in proximity is relatively long (where both distance factor D and time factor T tend to the value of one, interaction strength factor S also tends to the value of one. When the distance between users is relatively long and the time in which the users are in proximity is relatively short, each of distance factor D, time factor T, and interaction strength factor S tend to the value of zero. When the distance between users is relatively short and the time in which the users are in proximity is relatively short (where distance factor D tends to the value of one and time factor T tends to the value of zero), interaction strength factor S tends to the value of one half When the distance between users is relatively long and the time in which the users are in proximity is relatively long (where distance factor D tends to the value of zero and time factor T tends to the value of one), interaction strength factor S tends to the value of one half. When the distance between users equals d₀ and the time in which the users are in proximity equals to (where both distance factor D and time factor T are the value of one half), interaction strength factor S equals the value of one half. For all other scenarios, interaction strength factor S can be any value between zero and one depending on the values of distance factor D and time factor T.

In accordance with an embodiment, each of IPAS applications 308A and 308B are configured to determine interaction strength factor S in accordance with Equations 6-8 and provides interaction strength factor S to database 404. In accordance with another embodiment, database 404 determines interaction strength factor S based on the connection times and distances determined by IPAS Applications 308A and 308B. In either scenario, database 404 may store interaction strength factor S in records corresponding to computing device 302A and 302B (i.e., records 408 and 410). For example, as shown in FIG. 6, database 404 stores determined interaction strength factor S values in interaction strength factor fields (“S”) of records 408 and 410.

Database 404 may update risk level identifiers for the users maintained thereby based on the determined interaction strength factor S. In accordance with an embodiment, risk level identifiers may be determined as follows: If the interaction strength factor S between two users is less than 0.25, the risk level identifier may be set to a first value (e.g., Blue”), which indicates that no significant exposure occurred to anyone known to be infected. If the interaction strength factor S between two users is between 0.25 and 0.5 (and one of the user's risk level identifier is “Black”), the risk level identifier may be set to a second value (e.g., “Yellow”), which indicates that a user has been proximate to someone recently tested to be infected with a particular disease, and therefore, the user is at risk of being infected. If the interaction strength factor S between two users is between 0.5 and 0.75 (and one of the user's risk level identifier is “Black”), the risk level identifier may be set to a third value (e.g., “Orange”), which indicates that a user has been in significant proximity to someone recently tested to be infected with a particular disease. If the interaction strength factor S between two users is between 0.75 and 1 (and one of the user's risk level identifier is “Black”), the risk level identifier may be set to a third value (e.g., “Red”), which indicates that a user has close, prolonged contact with someone recently tested to be infected with a particular disease. It is noted that the values and color-coded identifiers are purely exemplary and that other values and identification schemes may be utilized to determine and/or designate risk level identifiers.

In the example shown in FIG. 6, the interaction strength factor S between the users associated with records 408 and 410 is 0.58. Accordingly, the risk level identifier determined for the user associated with record 410 is determined to be the third value (e.g., “Orange”). Accordingly, database 404 updates the risk level identifier stored in the risk level identifier field of record 410 to specify the value “Orange.” This is reflected in FIG. 7.

A user's risk level identifier may also be updated at a point-of-care (PoC) facility (e.g., a doctor's office, a testing facility, etc.). A user may visit a PoC facility to obtain a test to determine whether the person has a particular disease, to obtain a vaccine for a particular disease, obtain a test to determine whether the person has antibodies that counteract a particular disease, etc. Upon determining the result of the test or after administration of the vaccine, a healthcare provider that administers the test may update the risk level identifier field of the record associated with that user.

For example, FIG. 8 depicts a system 800 for updating a risk level identifier at a PoC facility in accordance with an example embodiment. As shown in FIG. 8, system 800 comprises a computing device 802 and a PoC computing device 816 that are communicatively coupled via a network 806. Computing device 802 is an example of computing devices 302A and 302B, as described above with reference to FIG. 3. Network 806 is an example of network 406, as shown in FIG. 4. PoC computing device 816 may comprise a smart phone, a tablet, a cell phone, a personal data assistant, a desktop or laptop computer, etc. In accordance with an embodiment, PoC computing device 816 may be a standalone device that is specifically configured to execute a PoC IPAS application 814, which is described below.

As shown in FIG. 8, computing device 802 comprises a transceiver 804 and an IPAS application 808, which is associated with a unique identifier. Transceiver 804 is an example of transceivers 304A and 304B, IPAS application 808 is an example of IPAS applications 308A and 308B, and unique identifier 810 is an example of unique identifiers 310A an 310B, as respectively described above with reference to FIGS. 3 and 4.

PoC computing device 816 comprises a transceiver 813 and PoC IPAS application 814. Transceiver 813 may be configured to operate in a similar fashion as transceiver 804. PoC IPAS application 814 is associated with a PoC unique identifier 812, which may be assigned to PoC IPAS application 814 at the time PoC IPAS application 814 is provisioned to and/or downloaded by PoC computing device 816 (in a similar fashion to how IPAS application 808 is assigned unique identifier 810).

PoC IPAS application 814 is configured to obtain unique identifier 810 from IPAS application 808. For instance, PoC computing device 816 and computing device 802 may establish a communication link (e.g., as described above with reference to FIG. 3). Once the link is established, IPAS application 808 may transmit unique identifier 810 to PoC unique identifier 812 via the communication link. IPAS application 808 may push unique identifier 810 to PoC IPAS application 814 upon establishing the communication link, or alternatively, may provide unique identifier 810 responsive to receiving a request from PoC IPAS application 814.

After a test or vaccine has been administered, a provider (e.g., a doctor, a nurse, a technician, etc.) at the PoC facility may utilize PoC IPAS application 814 to update a risk level identifier for the user. For instance, if the person tests positive for a particular disease, the provider may utilize PoC IPAS application 814 to set that person's risk level identifier to a first value (e.g., “Black”), which indicates that the person has tested positive for the particular disease. In another example, if the person tests positive for the antibodies, tests negative for the disease and/or receives a vaccine for the disease, the provider may utilize PoC IPAS application 814 to set that person's risk level identifier to a second value (e.g., “Green”), which indicates that the person has not been infected with the particular disease (e.g., the coronavirus), is immune to the disease, and/or that the user has been vaccinated.

To update the risk level identifier for the person, the provider may provide a command (e.g., via a graphical user interface provided by PoC IPAS application 814) that causes PoC IPAS application 814 to provide a request 818 to database 804 via network 806. Request 818 may be transmitted to database 804 by transceiver 813. Request 818 may comprise PoC unique identifier 812, unique identifier 810 and the risk level identifier value specified via the GUI.

Responsive to receiving request 818, database 804 may first determine whether request 818 originated from an authorized PoC facility. For example, database 804 may compare PoC unique identifier 812 to a list of PoC unique identifiers corresponding to authorized PoC facilities. The list may be maintained by database 804 and/or retrieved from a government agency. If a match is not found, then request 818 is denied and the risk level identifier for the person is not updated. However, if a match is found, then database 804 utilizes unique identifier 810 (as included in request 818) to access the person's record and update the risk level identifier field of that record with the risk level identifier value included in request 818. For example, as shown in FIG. 8, database 804 accesses record 820 corresponding to unique identifier 810 (“ef871089acd98104”) and updates the risk level identifier field of record 820. In this example, the person was found to be infected with the disease. Accordingly, the risk level identifier field of record 820 is updated with the value “Black.” The other fields of record 820 are not shown for the sake of brevity.

After setting the risk level identifier, database 804 may update the risk identifier fields for other users that were determined to be in proximity with the person, as well as updating the risk identifier fields for the users that were in proximity with the other users. For example, FIG. 9 depicts a plurality of records 902, 904, and 906 of a database 900 in accordance with an example embodiment. Database 900 is an example of database 804, as described above with reference to FIG. 8.

As shown in FIG. 9, the user associated with record 902 was just tested positive for a disease, and a healthcare provider has set his risk level identifier set to “Black.” As further shown in FIG. 9, there were three users that were determined to be in proximity with the user. These users are identified by the unique identifiers stored in the detected identifiers field (i.e., “b4278fbd4b108583”, “04b3bca029df5ea8”, and “a471070351f99218”). Accordingly, database 900 analyzes the interaction strength factor S for each of these users and determines whether the risk level identifiers for these users should also be changed.

In accordance with an embodiment, database 900 may also analyze the date/time of connection with each of the users to determine whether their respective risk level identifier flags are to be set. For example, database 900 may only analyze records having a date/time connection time that is within a predetermined time period corresponding to the infection/contagious period of the disease (e.g., 14 days).

In the example shown in FIG. 9, suppose that the date on which the risk level identifier for the user was set to “Black” on March 22, 2021. The user corresponding to unique identifier “b4278fbd4b108583” has a data/time of connection that is within the infection period (i.e., a date that is within 14 days from being diagnosed). Accordingly, database 900 analyzes the infection strength factor S for that user. In the example shown in FIG. 9, the infection strength factor S is 0.85. As described above, because the infection strength factor S is between 0.75 and 1, database 900 sets the risk level identifier for record 904 (which is associated with user having the unique identifier “b4278fbd4b108583”) to “Red,” which indicates that he had close, prolonged contact with someone recently tested to be infected with a particular disease.

Database 900 then analyzes the next user identified in the detected identifiers field for record 902. The user corresponding to unique identifier “04b3bca029df5ea8” has a data/time of connection that is within the infection period (i.e., a date that is within 14 days from being diagnosed). Accordingly, database 900 analyzes the infection strength factor S for that user. In the example shown in FIG. 9, the infection strength factor S is 0.53. As described above, because the infection strength factor S is between 0.50 and 0.75, database 900 sets the risk level identifier for record 906 (which is associated with user having the unique identifier “04b3bca029df5ea8”) to “Orange,” which indicates that he has been in significant proximity to someone recently tested to be infected with the disease.

Database 900 then analyzes the next user identified in the detected identifiers field for record 902. The user corresponding to unique identifier “a471070351f99218” does not have a date/time of connection that is within the infection period (i.e., the data/time of connection was more than 14 days ago). Accordingly, database 900 may not analyze the infection strength factor S for that user and may not update the risk level identifier for that user.

Database 900 also determines whether the users identified in the detected identifiers field for records 904 and 906 should have their risk level identifiers updated in a similar manner as described above. Each of the IPAS applications (e.g., IPAS application 808) associated with the users may periodically query database 900 to determine the risk level identifier associated with the user. The IPAS application may output an alert based on the determined risk level. For example, the IPAS application may cause an audio signal to be played back by a speaker of the computing device on which the IPAS application executes. The audio signal alerts the first user based on the received second risk level identifier. A different audio signal may be utilized for each of the determined risk level identifiers. In another example, the IPAS application may activate a vibration motor of the computing that that causes the first computing device to vibrate in accordance with a predetermined vibration pattern. The vibration pattern may be selected based on the determined risk level identifier. In a further example, the IPAS application may cause the computing device to display an alert notification on a display screen of the computing device based on the received second risk level identifier. For example, responsive to determining that the risk level identifier for a particular user is “Yellow,” the alert notification may display “You have been proximate to someone who was in (YELLOW, ORANGE, or RED) proximity of someone just tested to be COVID-19 positive so watch for symptoms and if they develop get tested.” Responsive to determining that the risk level identifier for a particular user is “Orange,” the alert notification may display, “You have been in close or long proximity to someone who was in (YELLOW, ORANGE, or RED) proximity to someone just tested to be COVID-19 positive so get tested as soon as possible.” Responsive to determining that the risk level identifier for a particular user is “Red,” the alert notification may display “You have had close, prolonged contact with someone in (YELLOW, ORANGE, or RED) proximity to someone just tested to be COVID-19 positive and may now be infected so it is imperative to self-quarantine and get tested as soon as possible.” In yet another example, the IPAS application may provide a visual alert based on the received second risk level identifier. For example, the IPAS application may activate one or more light emitting diodes (LEDs) or other type of light source of the computing device in accordance with a predetermined optical pattern (e.g., causing a light source to flash a predetermined number of times). The optical pattern may be selected based on the determined risk level identifier. In another example, the IPAS application may activate the display screen of the computing device and/or display and render a visual alert via the display screen. It is noted that the alerting techniques described above are purely exemplary and that other techniques may be utilized.

In accordance with the foregoing process, a contact chain between various users that were proximate to each other is determined and each of the users in the contact chain may have their risk level identifiers updated. The power of this approach is how widespread the alerting scheme is. For example, if each record for a particular user is associated with two other users that were in proximity the user, and the records for each of those two other users has their risk level identifiers updated, there would be a total of 2^{k} alerts issued, where k is the depth of the chain. If k is set to 14, then 16,384 alerts would be issued throughout the chain.

Another approach is to send the running average of the interaction strength factor through the chain (e.g., an average of interaction strength factors between users A and B users B and C, and users C and D). This would make the messaging simpler and more direct, but it hides the urgency of an earlier red flag alert. Again, these are by way of example, as there are many possible approach implementations to convey infection risk down the alert chain.

In accordance with an embodiment, a provision is put in place for self-diagnosis, e.g., via a home test (or self-test) kit. If a user takes the test, and the test indicates that the user is tested positive, the user may utilize the IAPS application to update his risk level identifier. For example, the IAPS application may provide a GUI by which the user may specify his test result. The IAPS application may update the risk level identifier of the user based on the result. For example, if the result is positive, the IAPS application may provide a request to the database. The request specifies that the unique identifier of the user and the user's test result. In response, the database retrieves the user's record using the unique identifier and sets the risk level identifier to “Black.” If the result is negative, the database sets the risk level identifier to “Green.”

In the event that the risk level identifier is set to “Black,” the database performs the contact alert process, as described above, where interaction strength factors for users that were determined to be proximate to the user are analyzed to determine whether the risk level identifiers for the users should be updated. If the recently-tested positive user was to venture out while being infected (e.g., during the 14-day quarantine period), another person determined to be proximate to the infected person would receive an alert in accordance with the embodiments described herein.

In accordance with an embodiment, after expiration of the quarantine period, database 900 automatically updates the risk level identifier of the user (e.g., the database changes it to “Green”). Any users that were detected to be proximate may also have their risk level identifiers updated accordingly if the dates/times of connection are more than the quarantine period.

In accordance with an embodiment, a record for a user comprises additional information, such as GPS coordinates of the user and/or contact information of the user (e.g., the user's phone number). Such information may be provided by the user's computing device via requests sent to the database (e.g., requests 406A and 406B). In accordance with such embodiments, the GPS coordinates and/or the dates/times of connections may be utilized by a government agency to generate infection heat maps. The contact information may be utilized by the database to push alert notifications to IPAS applications (rather than having IPAS applications periodically poll the database). However, such information would result in lesser privacy for the users and increase network traffic.

The following scenario demonstrates the foregoing features. Suppose that three days ago, Person A (an asymptomatic carrier) infects Person B. Two days ago, Person A infects person C, and Person B infects Person D. One day ago, Person A infects Person E, Person B infects Person F, Person C infects Person G, and Person D infects Person H. Person B decides to get tested and tests positive. The healthcare provider would utilize PoC IPAS application 814 to send a request to the database 804 to update Person B's risk level identifier to “Black”, as described above with reference to FIG. 8. This update triggers database 804 to update the risk level identifiers of other users that are in Person B's contact chain. For example, Person B's record would indicate that Persons A, D, and F were proximate to Person B. Accordingly, database 804 would update the risk level identifiers for Persons A, D, and F in accordance with the interaction strength factors for these users. Person A's record would indicate that Persons B, C, and E (and possibly other users) were proximate to Person A. Accordingly, database 804 would update the risk level identifiers for Persons B, C, and E (and the other users) in accordance with the interaction strength factors for these users (in a similar fashion as described above with reference to FIG. 9). Person C's record would indicate that Person G (and possibly other users) were proximate to Person C. Accordingly, the database would update the risk level identifiers for Person G (and the other users) in accordance with the interaction strength factors for these users (in a similar fashion as described above with reference to FIG. 9). Person D's record would indicate that Person H (and possibly other users) were proximate to Person D. Accordingly, the database would update the risk level identifiers for Person H (and the other users) in accordance with the interaction strength factors for these users (in a similar fashion as described above with reference to FIG. 9).

The IPAS application for all these users would periodically query the database and determine that their risk level identifiers have been updated. Assuming that the interaction strength factors were relatively strong between these users, each of these users would receive an alert to get themselves tested and/or self-quarantine. Upon quarantining, the spread of the disease is stopped.

After expiration of the quarantine period, the users have recovered and have developed antibodies, thereby rendering them immune (at least temporarily) to the disease. The database, after expiration of the quarantine period (or after the users receive a positive antibody test), updates the risk level identifiers for these users to “Green.”

In accordance with an embodiment, a business or organization (e.g., a concert hall, a grocery store, a baseball stadium, etc.) may utilize a computing device configured to execute an IPAS application (as described herein). As users, patrons, visitors, etc., approach the business or organization, the computing device communicates with the IPAS applications executing on such users' devices. The business' IPAS application may obtain the unique identifiers for such users from their respective IPAS applications and provide the unique identifiers to the database. The database may retrieve the records of such users and determine the risk level identifiers for such users. The risk level identifiers are returned to the business' IPAS applications, which display the risk level identifiers. The worker at the business or organization may deny entry to users having risk level identifiers that are not “Green” (or the like) and may allow entry to users having risk level identifiers that are “Green” (or the like).

In accordance with an embodiment, the database may maintain a whitelist of users for each of the users in the database. The whitelist would include users that have been proximate to the user and that have been vaccinated (e.g., such users would have risk level identifiers that are “Green” or the like). When the IPAS application of a user establishes a communication link with a user in the whitelist, the IPAS application stops tracking the connection time between the devices of the users, stops determining the distances between such devices, and/or terminates the communication link between the devices. This advantageously reduces the power utilized by the computing devices, thereby conserving valuable compute resources (e.g., processing cycles, memory, power, etc.).

Accordingly, a user may be alerted if they are in proximity to a person infected with a particular disease in many ways. For example, FIG. 10 shows a flowchart 1000 of a method performed by a first computing device for alerting a first user in accordance with an example embodiment. In an embodiment, flowchart 1000 may be implemented by IPAS application 308A or IPAS application 308B, as described above with reference to FIGS. 3 and 4, although the method is not limited to that implementation. Accordingly, flowchart 1000 will be described with continued reference to FIGS. 3 and 4. Other structural and operational embodiments will be apparent to persons skilled in the relevant art(s) based on the discussion regarding flowchart 1000 and systems 300 and 400 of FIGS. 3 and 4.

Flowchart 1000 begins with step 1002. In step 1002, a detection is made that a second computing device associated with a second user is proximate to the first computing device. For example, with reference to FIG. 3, IPAS application 308A detects that computing device 302B is proximate to computing device 302A, and IPAS application 308B detects that computing device 302A is proximate to computing device 302B. For example, each of computing devices 302A and 302B may periodically transmit a beacon signal 306A and 306B, respectively. IPAS application 308A detects that computing device 302B is proximate to computing device 302A responsive to detecting beacon signal 306B. IPAS application 308B detects that computing device 302A is proximate to computing device 302B responsive to detecting beacon signal 306A.

At step 1004, a communication link is established with the second computing device. For example, with reference to FIG. 3, computing device 302A establishes a communication link (e.g., a peer-to-peer network, a personal area network, etc.) with second computing device 302B.

At step 1006, a first identifier that uniquely identifies the second computing device and the second user is received from the second computing device via the communication link. For example, as shown in FIG. 3, IPAS application 308A transmits unique identifier 310A (that uniquely identifies computing device 302A and the user thereof) to IPAS application 308B via the communication link.

At step 1008, the first identifier and a second identifier that uniquely identifies the first computing device and the first user is provided to a database. The database maintains a first record associated with the first user and maintains a second record associated with the second user. The first record is associated with a first risk level identifier indicative of a risk that the first user has been infected with a particular disease. The second record is associated with a second risk level identifier indicative of a risk that the second user has been infected with the particular disease. For example, with reference to FIG. 4, IPAS application 308B provides request 406B to database 404. Request 406B comprises unique identifier 310A and unique identifier 310B (which uniquely identifies computing device 302B and the user thereof). Database 404 maintains record 410 associated with a first user and maintains record 408 associated with a second user. Record 410 comprises a risk level identifier field (“Flag”) that is indicative of a risk that the first user has been infected with a particular disease. Record 408 comprises a risk level identifier field that is indicative of a risk that the second user has been infected with the particular disease.

In step 1010, the second risk level identifier is received from the database. For example, with reference to FIG. 4, database 404 provides response 412B comprising the second risk level identifier (i.e., “Black”). Response 412B is received by IPAS application 308B.

In step 1012, an alert based at least on the received second risk level identifier is outputted via the first computing device. For example, with reference to FIG. 4, IPAS application 308B outputs an alert based at least on the received second risk level identifier.

In accordance with one or more embodiments, the alert may be outputted by causing an audio signal to be played back by a speaker of the first computing device, the audio signal alerting the first user based on the received second risk level identifier, activating a vibration motor of the first computing device that causes the first computing device to vibrate in accordance with a predetermined vibration pattern selected based on the received second risk level identifier, activating a light source of the computing device in accordance with a predetermined optical pattern selected based on the received second risk level identifier. and/or causing the computing device to display an alert notification on a display screen of the first computing device based on the received second risk level identifier. For example, with reference to FIG. 4, IPAS application 308B may cause an audio signal to be played back by speaker 418B of computing device 302B. A different audio signal may be played back based on the risk level identifier received from database 404. For example, if the risk level identifier is “Black,” a first audio signal may be played back. If the risk level identifier is “Orange,” a second audio signal may be played back. In addition to or in lieu of playing back different audio signals, the volume at which the audio signal is played back may vary based on the risk level identifier. In another example, IPAS application 308B may send a command to a vibration motor (not shown) of computing device 302B that causes computing device 302B to vibrate in accordance with a predetermined vibration pattern selected based on the risk level identifier received from database 404. For example, computing device 302B may store a plurality of different vibration patterns and may select the pattern to be utilized based on the risk level identifier. In a further example, IPAS application 308B may cause computing device 302B to display an alert notification on a display screen (not shown) of computing device 302B based on the risk level identifier received from database 404. In yet another example, the IPAS application may provide a visual alert based on the received second risk level identifier. For example, the IPAS application may activate one or more light emitting diodes (LEDs) or other type of light source of the computing device in accordance with a predetermined optical pattern (e.g., causing a light source to flash a predetermined number of times). The optical pattern may be selected based on the determined risk level identifier. In another example, the IPAS application may activate the display screen of the computing device and/or display and render a visual alert via the display screen.

In accordance with one or more embodiments, it is periodically determined whether the second risk level identifier has been updated by the database. Responsive to determining that the second risk level identifier has been updated, a second alert is outputted via the first computing device. For example, with reference to FIG. 4, IPAS application 308B may periodically provide a request to database 404 via transceiver 304B and network 406. The request comprises unique identifier 310B. Database 404 retrieves the record associated with unique identifier 310B (i.e., record 410) and provides a response to IPAS application 308B via network 406 and transceiver 304B. The response comprises the risk level identifier stored in the risk level identifier field of record 408. In the event that the risk level identifier indicates that the user has been exposed to someone that has been infected or may have been infected with a particular disease, IPAS application 308B issues an alert to the user via computing device 302B accordingly.

In accordance with one or more embodiments, the database is configured to update the second risk level identifier by determining that a third risk level identifier for a third record associated with the first record has been updated and updating the first risk level identifier associated with the first record based on the third risk level identifier. For example, with reference to FIG. 9, responsive to database 900 determining that the risk level identifier of record 902 has been updated to “Black”, database 900 determines whether other users that were proximate to the user associated with record 902 (as identified by the detected identifiers field of record 902) should have their risk level identifiers updated. For instance, database 900 analyzes the interaction strength factor S and/or date/time of connection with each of the other users. Database 900 updates the risk level identifiers for those other users based on the interaction strength factor S and/or date/time of connection. For example, as shown in FIG. 9, database 900 updates the risk level identifier field of record 904 (associated with a first user that was proximate to the user associated with record 902) to specify a risk level of “Red.” Database 900 also updates the risk level identifier field of record 906 (associated with a second user that was proximate to the user associated with record 902) to specify a risk level of “Orange.”

In accordance with one or more embodiments, the alert that is outputted is based on a determined distance between the computing devices and a determined duration in which the computing devices were connected via the communication link. For example, FIG. 11 shows a flowchart 1100 of a method for alerting a user based on a determined distance between the first computing device and the second computing device and a determined duration in which the first computing device and the second computing device were connected via a communication link in accordance with an example embodiment. In an embodiment, flowchart 1100 may be implemented by IPAS application 308A or IPAS application 308B, as described above with reference to FIGS. 3 and 4, although the method is not limited to that implementation. Accordingly, flowchart 1100 will be described with continued reference to FIGS. 3 and 4. Other structural and operational embodiments will be apparent to persons skilled in the relevant art(s) based on the discussion regarding flowchart 1100 and systems 300 and 400 of FIGS. 3 and 4.

Flowchart 1100 begins with step 1102. In step 1102, a distance between the first computing device and the second computing device is determined. For example, with reference to FIG. 4, IPAS application 308B determines the distance between computing devices 302B and 302A.

In accordance with one or more embodiments, IPAS application 308B determines the distance by generating an audio signature signal that is unique to the first computing device, transmitting the audio signature signal, determining a first time at which the audio signature signal was transmitted, receiving a response signal from the second computing device, determining a second time at which the response signal was received, and determining the distance based on the determined first time and the determined second time. For example, with reference to FIG. 4, audio signature signal generator 416B generates audio signature signal 420B that is unique to computing device 302B. For instance, audio signature signal generator 416B may utilize communication parameters that are specific to computing device 302B to generate and/or transmit audio signature signal 420B via speaker 418B of computing device 302B. The communication parameters (e.g., a frequency at which audio signature signal 420B is transmitted, an audio pattern to be utilized to transmit audio signature signal 420B, etc.) may be based on unique identifier 310B. For instance, audio signature signal generator 416B may comprise circuitry, logic, etc., that receives unique identifier 310B as an input and outputs the communication parameters based on unique identifier 310B. IPAS application 308B may initiate a timer at the time transmission of audio signature signal 420B is initiated. IPAS application 308A may provide a response signal 426A to IPAS application 308B via the communication link established therebetween, which indicates to IPAS application 308B that IPAS application 308A detected audio signature signal 420B. IPAS application 308B then stops the timer to record the time t_{d}, which represents the time it took audio signature signal 420B to travel from computing device 302B to computing device 302A. The time to may be based on a first time at which the timer is initiated and a second time at which the timer is stopped. For example, the time to may be determined by subtracting the first time from the second time. IPAS application 308B may determine the distance between computing devices 302B and 302A based on time to in accordance with Equation 5 described above. Alternatively, IPAS application 308B may provide time t_{d} to database 404, which determines the distance in accordance with Equation 5.

In accordance with one or more embodiments, communication parameters by which the audio signature signal is transmitted by the first computing device are provided to the second computing device via the communication link. The second computing device is configured to detect the audio signature signal in accordance with the communication parameters provided thereto. For example, with reference to FIG. 4, IPAS application 308B provides the communication parameters to IPAS application 308A.

At step 1104 a duration in which the first computing device and the second computing device were connected via the communication link is determined. For example, with reference to FIG. 4, upon establishing a communication link between computing devices 302A and 302B, IPAS application 308B may initiate a timer. The timer is stopped after the connection or network between computing devices 302A and 302B is terminated (e.g., when computing devices 302A and 302B are no longer in range). The duration may be based on a first time at which the timer is initiated and a second time at which the timer is stopped. For example, the duration may be determined by subtracting the first time from the second time.

At step 1106, the determined distance and the determined duration are provided to the database, the database configured to update the second risk level identifier based on the first risk level identifier, the determined distance, and the determined duration. For example, with reference to FIG. 4, IPAS application 308B may provide requests 414B and/or 428B that comprise the determined distance and determined duration, respectively. Alternatively, a single request (e.g., one of request 414B or 428B) may comprise both the determined distance and the determined duration. It is noted that in an embodiment in which database 404 determines the distance, IPAS application 308B does not provide the determined distance to database 404. As shown in FIGS. 6 and 7, database 404 determines an interaction strength factor S based on the determined distance and the connection duration (as specified in the distance and connection duration fields). The interaction strength factor S and risk level identifier are utilized to determine a risk level identifier for the user associated with record 410. As shown in FIG. 7, record 410 is updated to include the risk level identifier of “Orange”.

### III. Example Mobile and Stationary Device Embodiments

The systems and methods described above, including the infected person alert systems and methods described in reference to FIGS. 2-4 and 6-11 may be implemented in hardware, or hardware combined with one or both of software and/or firmware. For example, computing devices 202A-202N, database 204, IPAS applications 208A-208N, computing devices 302A and 302B, transceivers 304A and 304B, IPAS applications 308A and 308B, audio signature signal generators 416A and 416B, database 404, computing devices 802, PoC computing device 816, transceiver 804, transceiver 813, IPAS application 808, PoC IPAS application 814, database 804, and/or database 900 and/or each of the components described therein, and flowcharts 1000 and/or 1100 may be each implemented as computer program code/instructions configured to be executed in one or more processors and stored in a computer readable storage medium. Alternatively, computing devices 202A-202N, database 204, IPAS applications 208A-208N, computing devices 302A and 302B, transceivers 304A and 304B, IPAS applications 308A and 308B, audio signature signal generators 416A and 416B, database 404, computing devices 802, PoC computing device 816, transceiver 804, transceiver 813, IPAS application 808, PoC IPAS application 814, database 804, and/or database 900 and/or each of the components described therein, and flowcharts 1000 and/or 1100 may be implemented as hardware logic/electrical circuitry. In an embodiment, computing devices 202A-202N, database 204, IPAS applications 208A-208N, computing devices 302A and 302B, transceivers 304A and 304B, IPAS applications 308A and 308B, audio signature signal generators 416A and 416B, database 404, computing devices 802, PoC computing device 816, transceiver 804, transceiver 813, IPAS application 808, PoC IPAS application 814, database 804, and/or database 900 and/or each of the components described therein, and flowcharts 1000 and/or 1100 may be implemented in one or more SoCs (system on chip). An SoC may include an integrated circuit chip that includes one or more of a processor (e.g., a central processing unit (CPU), microcontroller, microprocessor, digital signal processor (DSP), etc.), memory, one or more communication interfaces, and/or further circuits, and may optionally execute received program code and/or include embedded firmware to perform functions.

FIG. 12 shows a block diagram of an exemplary mobile device 1200 including a variety of optional hardware and software components, shown generally as components 1202. Any number and combination of the features/elements of computing devices 202A-202N, database 204, IPAS applications 208A-208N, computing devices 302A and 302B, transceivers 304A and 304B, IPAS applications 308A and 308B, audio signature signal generators 416A and 416B, database 404, computing devices 802, PoC computing device 816, transceiver 804, transceiver 813, IPAS application 808, PoC IPAS application 814, database 804, and/or database 900 may be implemented as components 1202 included in a mobile device embodiment, as well as additional and/or alternative features/elements, as would be known to persons skilled in the relevant art(s). It is noted that any of components 1202 can communicate with any other of components 1202, although not all connections are shown, for ease of illustration. Mobile device 1200 can be any of a variety of mobile devices described or mentioned elsewhere herein or otherwise known (e.g., cell phone, smartphone, handheld computer, Personal Digital Assistant (PDA), etc.) and can allow wireless two-way communications with one or more mobile devices over one or more communications networks 1204, such as a cellular or satellite network, or with a local area or wide area network.

The illustrated mobile device 1200 can include a controller or processor referred to as processor circuit 1210 for performing such tasks as signal coding, image processing, data processing, input/output processing, power control, and/or other functions. Processor circuit 1210 is an electrical and/or optical circuit implemented in one or more physical hardware electrical circuit device elements and/or integrated circuit devices (semiconductor material chips or dies) as a central processing unit (CPU), a microcontroller, a microprocessor, and/or other physical hardware processor circuit. Processor circuit 1210 may execute program code stored in a computer readable medium, such as program code of one or more applications 1214, operating system 1212, any program code stored in memory 1220, etc. Operating system 1212 can control the allocation and usage of the components 1202 and support for one or more application programs 1214 (a.k.a. applications, “apps”, etc.). Application programs 1214 can include common mobile computing applications (e.g., email applications, calendars, contact managers, web browsers, messaging applications) and any other computing applications (e.g., word processing applications, mapping applications, media player applications).

As illustrated, mobile device 1200 can include memory 1220. Memory 1220 can include non-removable memory 1222 and/or removable memory 1224. The non-removable memory 1222 can include RAM, ROM, flash memory, a hard disk, or other well-known memory storage technologies. The removable memory 1224 can include flash memory or a Subscriber Identity Module (SIM) card, which is well known in GSM communication systems, or other well-known memory storage technologies, such as “smart cards.” The memory 1220 can be used for storing data and/or code for running operating system 1212 and applications 1214. Example data can include web pages, text, images, sound files, video data, or other data sets to be sent to and/or received from one or more network servers or other devices via one or more wired or wireless networks. Memory 1220 can be used to store a subscriber identifier, such as an International Mobile Subscriber Identity (IMSI), and an equipment identifier, such as an International Mobile Equipment Identifier (IMEI). Such identifiers can be transmitted to a network server to identify users and equipment.

A number of programs may be stored in memory 1220. These programs include operating system 1212, one or more application programs 1214, and other program modules and program data. Examples of such application programs or program modules may include, for example, computer program logic (e.g., computer program code or instructions) for implementing the systems described above, including the embodiments described in reference to FIGS. 2-4 and 6-11.

Mobile device 1200 can support one or more input devices 1230, such as a touch screen 1232, microphone 1234, camera 1236, physical keyboard 1238 and/or trackball 1240 and one or more output devices 1250, such as a speaker 1252 and a display 1254.

Other possible output devices (not shown) can include piezoelectric or other haptic output devices. Some devices can serve more than one input/output function. For example, touch screen 1232 and display 1254 can be combined in a single input/output device. The input devices 1230 can include a Natural User Interface (NUI).

Wireless modem(s) 1260 can be coupled to antenna(s) (not shown) and can support two-way communications between processor circuit 1210 and external devices, as is well understood in the art. The modem(s) 1260 are shown generically and can include a cellular modem 1266 for communicating with the mobile communication network 1204 and/or other radio-based modems (e.g., Bluetooth 1264 and/or Wi-Fi 1262). Cellular modem 1266 may be configured to enable phone calls (and optionally transmit data) according to any suitable communication standard or technology, such as GSM, 3G, 4G, 5G, etc. At least one of the wireless modem(s) 1260 is typically configured for communication with one or more cellular networks, such as a GSM network for data and voice communications within a single cellular network, between cellular networks, or between the mobile device and a public switched telephone network (PSTN).

Mobile device 1200 can further include at least one input/output port 1280, a power supply 1282, a satellite navigation system receiver 1284, such as a Global Positioning System (GPS) receiver, an accelerometer 1286, and/or a physical connector 1290, which can be a USB port, IEEE 1394 (FireWire) port, and/or RS-232 port. The illustrated components 1202 are not required or all-inclusive, as any components can be not present and other components can be additionally present as would be recognized by one skilled in the art.

Furthermore, FIG. 13 depicts an exemplary implementation of a computing device 1300 in which embodiments may be implemented, including computing device 100, display 108, GUI 114, annotation engine 102, imaging system 110, camera(s) 106, user interface(s) 104, smart phone 200, annotation engine 400, feature point detector 402, surface detector 404, and/or smart phone 500, and/or each of the components described therein, and flowchart 300 and/or 600. The description of computing device 1300 provided herein is provided for purposes of illustration, and is not intended to be limiting. Embodiments may be implemented in further types of computer systems, as would be known to persons skilled in the relevant art(s).

As shown in FIG. 13, computing device 1300 includes one or more processors, referred to as processor circuit 1302, a system memory 1304, and a bus 1306 that couples various system components including system memory 1304 to processor circuit 1302. Processor circuit 1302 is an electrical and/or optical circuit implemented in one or more physical hardware electrical circuit device elements and/or integrated circuit devices (semiconductor material chips or dies) as a central processing unit (CPU), a microcontroller, a microprocessor, and/or other physical hardware processor circuit. Processor circuit 1302 may execute program code stored in a computer readable medium, such as program code of operating system 1330, application programs 1332, other programs 1334, etc. Bus 1306 represents one or more of any of several types of bus structures, including a memory bus or memory controller, a peripheral bus, an accelerated graphics port, and a processor or local bus using any of a variety of bus architectures. System memory 1304 includes read only memory (ROM) 1308 and random access memory (RAM) 1310. A basic input/output system 1312 (BIOS) is stored in ROM 1308.

Computing device 1300 also has one or more of the following drives: a hard disk drive 1314 for reading from and writing to a hard disk, a magnetic disk drive 1316 for reading from or writing to a removable magnetic disk 1318, and an optical disk drive 1320 for reading from or writing to a removable optical disk 1322 such as a CD ROM, DVD ROM, or other optical media. Hard disk drive 1314, magnetic disk drive 1316, and optical disk drive 1320 are connected to bus 1306 by a hard disk drive interface 1324, a magnetic disk drive interface 1326, and an optical drive interface 1328, respectively. The drives and their associated computer-readable media provide nonvolatile storage of computer-readable instructions, data structures, program modules and other data for the computer. Although a hard disk, a removable magnetic disk and a removable optical disk are described, other types of hardware-based computer-readable storage media can be used to store data, such as flash memory cards, digital video disks, RAMs, ROMs, and other hardware storage media.

A number of program modules may be stored on the hard disk, magnetic disk, optical disk, ROM, or RAM. These programs include operating system 1330, one or more application programs 1332, other programs 1334, and program data 1336. Application programs 1332 or other programs 1334 may include, for example, computer program logic (e.g., computer program code or instructions) for implementing the systems described above, including the embodiments described in reference to FIGS. 2-4 and 6-11.

A user may enter commands and information into the computing device 1300 through input devices such as keyboard 1338 and pointing device 1340. Other input devices (not shown) may include a microphone, joystick, game pad, satellite dish, scanner, a touch screen and/or touch pad, a voice recognition system to receive voice input, a gesture recognition system to receive gesture input, or the like. These and other input devices are often connected to processor circuit 1302 through a serial port interface 1342 that is coupled to bus 1306, but may be connected by other interfaces, such as a parallel port, game port, or a universal serial bus (USB).

A display screen 1344 is also connected to bus 1306 via an interface, such as a video adapter 1346. Display screen 1344 may be external to, or incorporated in computing device 1300. Display screen 1344 may display information, as well as being a user interface for receiving user commands and/or other information (e.g., by touch, finger gestures, virtual keyboard, etc.). In addition to display screen 1344, computing device 1300 may include other peripheral output devices (not shown) such as speakers and printers.

Computing device 1300 is connected to a network 1348 (e.g., the Internet) through an adaptor or network interface 1350, a modem 1352, or other means for establishing communications over the network. Modem 1352, which may be internal or external, may be connected to bus 1306 via serial port interface 1342, as shown in FIG. 13, or may be connected to bus 1306 using another interface type, including a parallel interface.

As used herein, the terms “computer program medium,” “computer-readable medium,” and “computer-readable storage medium” are used to generally refer to physical hardware media such as the hard disk associated with hard disk drive 1314, removable magnetic disk 1318, removable optical disk 1322, other physical hardware media such as RAMs, ROMs, flash memory cards, digital video disks, zip disks, MEMs, nanotechnology-based storage devices, and further types of physical/tangible hardware storage media (including system memory 1304 of FIG. 13). Such computer-readable storage media are distinguished from and non-overlapping with communication media (do not include communication media). Communication media typically embodies computer-readable instructions, data structures, program modules or other data in a modulated data signal such as a carrier wave. The term “modulated data signal” means a signal that has one or more of its characteristics set or changed in such a manner as to encode information in the signal. By way of example, and not limitation, communication media includes wireless media such as acoustic, RF, infrared and other wireless media, as well as wired media. Embodiments are also directed to such communication media.

As noted above, computer programs and modules (including application programs 1332 and other programs 1334) may be stored on the hard disk, magnetic disk, optical disk, ROM, RAM, or other hardware storage medium. Such computer programs may also be received via network interface 1350, serial port interface 1352, or any other interface type. Such computer programs, when executed or loaded by an application, enable computing device 1300 to implement features of embodiments discussed herein. Accordingly, such computer programs represent controllers of the computing device 1300.

Embodiments are also directed to computer program products comprising computer code or instructions stored on any computer-readable medium. Such computer program products include hard disk drives, optical disk drives, memory device packages, portable memory sticks, memory cards, and other types of physical storage hardware.

### IV. Further Example Embodiments

A method performed by a first computing device for alerting a first user is described herein. The method includes: detecting that a second computing device associated with a second user is proximate to the first computing device; establishing a communication link with the second computing device; receiving, from the second computing device, a first identifier that uniquely identifies the second computing device and the second user via the communication link; providing the first identifier and a second identifier that uniquely identifies the first computing device and the first user to a database that maintains a first record associated with the first user and maintains a second record associated with the second user, the first record being associated with a first risk level identifier indicative of a risk that the first user has been infected with a particular disease, the second record being associated with a second risk level identifier indicative of a risk that the second user has been infected with the particular disease; receiving the second risk level identifier from the database; and outputting, via the first computing device, an alert based at least on the received second risk level identifier.

In one implementation of the foregoing method, the method further comprises: determining a distance between the first computing device and the second computing device; determining a duration in which the first computing device and the second computing device were connected via the communication link; and providing the determined distance and the determined duration to the database, the database configured to update the second risk level identifier based on the first risk level identifier, the determined distance and the determined duration.

In one implementation of the foregoing method, determining the distance comprises: generating an audio signature signal that is unique to the first computing device; transmitting the audio signature signal; determining a first time at which the audio signature signal was transmitted; receiving a response signal from the second computing device; determining a second time at which the response signal was received; and determining the distance based on the determined first time and the determined second time.

In one implementation of the foregoing method, the method further comprises: providing, to the second computing device via the communication link, communication parameters by which the audio signature signal is transmitted by the first computing device, the second computing device configured to detect the audio signature signal in accordance with the communication parameters provided thereto.

In one implementation of the foregoing method, said outputting comprises at least one of: causing an audio signal to be played back by a speaker of the first computing device, the audio signal alerting the first user based on the received second risk level identifier; activating a vibration motor of the first computing device that causes the first computing device to vibrate in accordance with a predetermined vibration pattern selected based on the received second risk level identifier; activating a light source of the computing device in accordance with a predetermined optical pattern selected based on the received second risk level identifier; or causing the computing device to display an alert notification on a display screen of the first computing device based on the received second risk level identifier.

In one implementation of the foregoing method, the method further comprises: periodically determining whether the second risk level identifier has been updated by the database; and responsive to determining that the second risk level identifier has been updated, outputting, via the first computing device, a second alert based at least on the updated second risk level identifier.

In one implementation of the foregoing method, the database is configured to update the second risk level identifier by: determining that a third risk level identifier for a third record associated with a third user and associated with the first record has been updated; and updating the first risk level identifier associated with the first record based on the third risk level identifier.

In one implementation of the foregoing method, the third risk level identifier is updated by a provider at a point-of-care facility responsive to the third user testing positive for the particular disease.

In one implementation of the foregoing method, the third risk level identifier is updated by the third user responsive to the third user testing positive for the particular disease via a self-administered test.

A first computing device for alerting a first user in accordance with any of the embodiments described herein is also disclosed. The first computing device includes: at least one processor circuit; and at least one memory that stores program code configured to be executed by the at least one processor circuit, the program code comprising: an infected person alert system configured to: detect that a second computing device associated with a second user is proximate to the first computing device; establish a communication link with the second computing device; receive, from the second computing device, a first identifier that uniquely identifies the second computing device and the second user via the communication link; provide the first identifier and a second identifier that uniquely identifies the first computing device and the first user to a database that maintains a first record associated with the first user and maintains a second record associated with the second user, the first record being associated with a first risk level identifier indicative of a risk that the first user has been infected with a particular disease, the second record being associated with a second risk level identifier indicative of a risk that the second user has been infected with the particular disease; receive the second risk level identifier from the database; and output, via the first computing device, an alert based at least on the received second risk level identifier.

In one implementation of the foregoing first computing device, the infected person alert system further configured to: determine a distance between the first computing device and the second computing device; determine a duration in which the first computing device and the second computing device were connected via the communication link; and provide the determined distance and the determined duration to the database, the database configured to update the second risk level identifier based on the first risk level identifier, the determined distance and the determined duration.

In one implementation of the foregoing first computing device, the infected person alert system is further configured to: generate an audio signature signal that is unique to the first computing device; transmit the audio signature signal via a speaker of the first computing device; determine a first time at which the audio signature signal was transmitted; receive a response signal from the second computing device; determine a second time at which the response signal was received; and determine the distance based on the determined first time and the determined second time.

In one implementation of the foregoing first computing device, the infected person alert system is further configured to: provide, to the second computing device via the communication link, communication parameters by which the audio signature signal is transmitted by the first computing device, the second computing device configured to detect the audio signature signal in accordance with the communication parameters provided thereto.

In one implementation of the foregoing first computing device, the infected person alert system is configured to output the alert by: causing an audio signal to be played back by a speaker of the first computing device, the audio signal alerting the first user based on the received second risk level identifier; activating a vibration motor of the first computing device that causes the first computing device to vibrate in accordance with a predetermined vibration pattern selected based on the received second risk level identifier; activating a light source of the computing device in accordance with a predetermined optical pattern selected based on the received second risk level identifier; or causing the computing device to display an alert notification on a display screen of the first computing device based on the received second risk level identifier.

In one implementation of the foregoing first computing device, the infected person alert system is further configured to: periodically determine whether the second risk level identifier has been updated by the database; and responsive to determining that the second risk level identifier has been updated, output, via the first computing device, a second alert based at least on the updated second risk level identifier.

In one implementation of the foregoing first computing device, the database is configured to update the second risk level identifier by: determining that a third risk level identifier for a third record associated with a third user and associated with the first record has been updated; and updating the first risk level identifier associated with the first record based on the third risk level identifier.

A computer-readable storage medium having program instructions recorded thereon that, when executed by at least one processor of a first computing device, perform a method for alerting a first user. The method includes: detecting that a second computing device associated with a second user is proximate to the first computing device; establishing a communication link with the second computing device; receiving, from the second computing device, a first identifier that uniquely identifies the second computing device and the second user via the communication link; providing the first identifier and a second identifier that uniquely identifies the first computing device and the first user to a database that maintains a first record associated with the first user and maintains a second record associated with the second user, the first record being associated with a first risk level identifier indicative of a risk that the first user has been infected with a particular disease, the second record being associated with a second risk level identifier indicative of a risk that the second user has been infected with the particular disease; receiving the second risk level identifier from the database; and outputting, via the first computing device, an alert based at least on the received second risk level identifier.

In one implementation of the foregoing computer-readable storage medium, the method further comprising: determining a distance between the first computing device and the second computing device; determining a duration in which the first computing device and the second computing device were connected via the communication link; and providing the determined distance and the determined duration to the database, the database configured to update the second risk level identifier based on the first risk level identifier, the determined distance and the determined duration.

In one implementation of the foregoing computer-readable storage medium, determining the distance comprises: generating an audio signature signal that is unique to the first computing device; transmitting the audio signature signal; determining a first time at which the audio signature signal was transmitted; receiving a response signal from the second computing device; determining a second time at which the response signal was received; and determining the distance based on the determined first time and the determined second time.

In one implementation of the foregoing computer-readable storage medium, the method further comprises: providing, to the second computing device via the communication link, communication parameters by which the audio signature signal is transmitted by the first computing device, the second computing device configured to detect the audio signature signal in accordance with the communication parameters provided thereto.

In one implementation of the foregoing computer-readable storage medium, said outputting comprises at least one of: causing an audio signal to be played back by a speaker of the first computing device, the audio signal alerting the first user based on the received second risk level identifier; activating a vibration motor of the first computing device that causes the first computing device to vibrate in accordance with a predetermined vibration pattern selected based on the received second risk level identifier; or causing the computing device to display an alert notification on a display screen of the first computing device based on the received second risk level identifier.

In one implementation of the foregoing computer-readable storage medium, the method further comprises: periodically determining whether the second risk level identifier has been updated by the database; and responsive to determining that the second risk level identifier has been updated, outputting, via the first computing device, a second alert based at least on the updated second risk level identifier.

### V Conclusion

While various example embodiments have been described above, it should be understood that they have been presented by way of example only, and not limitation. It will be understood by those skilled in the relevant art(s) that various changes in form and details may be made therein without departing from the spirit and scope of the embodiments as defined in the appended claims. Accordingly, the breadth and scope of the disclosure should not be limited by any of the above-described example embodiments, but should be defined only in accordance with the following claims and their equivalents.

What is claimed is:

1. A method performed by a first computing device for alerting a first user, comprising:
detecting that a second computing device associated with a second user is proximate to the first computing device;
establishing a communication link with the second computing device;
receiving, from the second computing device, a first identifier that uniquely identifies the second computing device and the second user via the communication link;
providing the first identifier and a second identifier that uniquely identifies the first computing device and the first user to a database that maintains a first record associated with the first user and maintains a second record associated with the second user, the first record being associated with a first risk level identifier indicative of a risk that the first user has been infected with a particular disease, the second record being associated with a second risk level identifier indicative of a risk that the second user has been infected with the particular disease;
receiving the second risk level identifier from the database; and
outputting, via the first computing device, an alert based at least on the received second risk level identifier.

2. The method of claim 1, further comprising:
determining a distance between the first computing device and the second computing device;
determining a duration in which the first computing device and the second computing device were connected via the communication link; and
providing the determined distance and the determined duration to the database, the database configured to update the second risk level identifier based on the first risk level identifier, the determined distance and the determined duration.

3. The method of claim 2, wherein determining the distance comprises:
generating an audio signature signal that is unique to the first computing device;
transmitting the audio signature signal;
determining a first time at which the audio signature signal was transmitted;
receiving a response signal from the second computing device;
determining a second time at which the response signal was received; and
determining the distance based on the determined first time and the determined second time.

4. The method of claim 3, further comprising:
providing, to the second computing device via the communication link, communication parameters by which the audio signature signal is transmitted by the first computing device, the second computing device configured to detect the audio signature signal in accordance with the communication parameters provided thereto.

5. The method of claim 1, wherein said outputting comprises at least one of:
causing an audio signal to be played back by a speaker of the first computing device, the audio signal alerting the first user based on the received second risk level identifier;
activating a vibration motor of the first computing device that causes the first computing device to vibrate in accordance with a predetermined vibration pattern selected based on the received second risk level identifier;
activating a light source of the computing device in accordance with a predetermined optical pattern selected based on the received second risk level identifier; or
causing the computing device to display an alert notification on a display screen of the first computing device based on the received second risk level identifier.

6. The method of claim 1, further comprising:
periodically determining whether the second risk level identifier has been updated by the database; and
responsive to determining that the second risk level identifier has been updated, outputting, via the first computing device, a second alert based at least on the updated second risk level identifier.

7. The method of claim 6, wherein the database is configured to update the second risk level identifier by:
determining that a third risk level identifier for a third record associated with a third user and associated with the first record has been updated; and
updating the first risk level identifier associated with the first record based on the third risk level identifier.

8. The method of claim 7, wherein the third risk level identifier is updated by a provider at a point-of-care facility responsive to the third user testing positive for the particular disease.

9. The method of claim 7, wherein the third risk level identifier is updated by the third user responsive to the third user testing positive for the particular disease via a self-administered test.

10. A first computing device for alerting a first user, comprising:
at least one processor circuit; and
at least one memory that stores program code configured to be executed by the at least one processor circuit, the program code comprising:
an infected person alert system configured to:
detect that a second computing device associated with a second user is proximate to the first computing device;
establish a communication link with the second computing device;
receive, from the second computing device, a first identifier that uniquely identifies the second computing device and the second user via the communication link;
provide the first identifier and a second identifier that uniquely identifies the first computing device and the first user to a database that maintains a first record associated with the first user and maintains a second record associated with the second user, the first record being associated with a first risk level identifier indicative of a risk that the first user has been infected with a particular disease, the second record being associated with a second risk level identifier indicative of a risk that the second user has been infected with the particular disease;
receive the second risk level identifier from the database; and
output, via the first computing device, an alert based at least on the received second risk level identifier.

11. The first computing device of claim 10, the infected person alert system further configured to:
determine a distance between the first computing device and the second computing device;
determine a duration in which the first computing device and the second computing device were connected via the communication link; and
provide the determined distance and the determined duration to the database, the database configured to update the second risk level identifier based on the first risk level identifier, the determined distance and the determined duration.

12. The first computing device of claim 11, wherein the infected person alert system is further configured to:
generate an audio signature signal that is unique to the first computing device;
transmit the audio signature signal via a speaker of the first computing device;
determine a first time at which the audio signature signal was transmitted;
receive a response signal from the second computing device;
determine a second time at which the response signal was received; and
determine the distance based on the determined first time and the determined second time.

13. The first computing device of claim 12, wherein the infected person alert system is further configured to:
provide, to the second computing device via the communication link, communication parameters by which the audio signature signal is transmitted by the first computing device, the second computing device configured to detect the audio signature signal in accordance with the communication parameters provided thereto.

14. The first computing device of claim 10, wherein the infected person alert system is configured to output the alert by:
causing an audio signal to be played back by a speaker of the first computing device, the audio signal alerting the first user based on the received second risk level identifier;
activating a vibration motor of the first computing device that causes the first computing device to vibrate in accordance with a predetermined vibration pattern selected based on the received second risk level identifier;
activating a light source of the computing device in accordance with a predetermined optical pattern selected based on the received second risk level identifier; or
causing the computing device to display an alert notification on a display screen of the first computing device based on the received second risk level identifier.

15. The first computing device of claim 10, wherein the infected person alert system is further configured to:
periodically determine whether the second risk level identifier has been updated by the database; and
responsive to determining that the second risk level identifier has been updated, output, via the first computing device, a second alert based at least on the updated second risk level identifier.

16. The first computing device of claim 15, wherein the database is configured to update the second risk level identifier by:
determining that a third risk level identifier for a third record associated with a third user and associated with the first record has been updated; and
updating the first risk level identifier associated with the first record based on the third risk level identifier.

17. A computer-readable storage medium having program instructions recorded thereon that, when executed by at least one processor of a first computing device, perform a method for alerting a first user, the method comprising:
detecting that a second computing device associated with a second user is proximate to the first computing device;
establishing a communication link with the second computing device;
receiving, from the second computing device, a first identifier that uniquely identifies the second computing device and the second user via the communication link;
providing the first identifier and a second identifier that uniquely identifies the first computing device and the first user to a database that maintains a first record associated with the first user and maintains a second record associated with the second user, the first record being associated with a first risk level identifier indicative of a risk that the first user has been infected with a particular disease, the second record being associated with a second risk level identifier indicative of a risk that the second user has been infected with the particular disease;
receiving the second risk level identifier from the database; and
outputting, via the first computing device, an alert based at least on the received second risk level identifier.

18. The computer-readable storage medium of claim 17, the method further comprising:
determining a distance between the first computing device and the second computing device;
determining a duration in which the first computing device and the second computing device were connected via the communication link; and
providing the determined distance and the determined duration to the database, the database configured to update the second risk level identifier based on the first risk level identifier, the determined distance and the determined duration.

19. The computer-readable storage medium of claim 18, wherein determining the distance comprises:
generating an audio signature signal that is unique to the first computing device;
transmitting the audio signature signal;
determining a first time at which the audio signature signal was transmitted;
receiving a response signal from the second computing device;
determining a second time at which the response signal was received; and
determining the distance based on the determined first time and the determined second time.

20. The computer-readable storage medium of claim 15, the method further comprising:
providing, to the second computing device via the communication link, communication parameters by which the audio signature signal is transmitted by the first computing device, the second computing device configured to detect the audio signature signal in accordance with the communication parameters provided thereto.
